(19) United States
(12) Patent Application Publication
(10) Pub. No.: US 2012/0115951 A1
(43) Pub. Date: May 10, 2012

(54) PROTECTIVE ACTION OF SEROFENDIC ACID ON CARDIAC MUSCLE

(75) Inventors: Masaharu Akao, Kyoto (JP); Akinori Akaike, Kyoto (JP); Toru Kita, Kyoto (JP); Yasuki Kihara, Kyoto (JP)

(73) Assignee: Eisai R & D Managment Co., Ltd, Tokyo (JP)

(21) Appl. No.: 12/066,107

(22) PCT Filed: Sep. 8, 2006
(86) PCT No.: PCT/JP2006/318326

§ 371 Date: Sep. 22, 2009

(30) Foreign Application Priority Data
Sep. 8, 2005 (JP) 2005 260205

(51) Int. Cl. A61K 31/19; C07C 323/53; A61P 9/04; A61K 31/10; A61P 9/10; A61P 9/00; C07C 325/02; C07C 317/46

(52) U.S. Cl. 514/557; 562/403; 568/20; 514/708

(57) ABSTRACT

A cardiac myocyte protective agent, comprising a compound represented by the formula (Ia) below, a pharmaceutically acceptable salt thereof, or a solvate thereof:

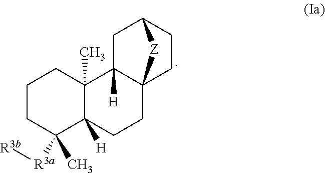

This application is the U.S. national phase application under 35 U.S.C. §371 of International Patent Application No. PCT/JP2006/318326 filed Sep. 8, 2006, which claims the benefit of priority to Japanese Patent Application No. 2005-260205 filed Sep. 8, 2005, the disclosures of all of which are hereby incorporated by reference in their entireties. The International Application was published in Japanese on Mar. 15, 2007 as WO 2007/029889.

### FIELD OF THE INVENTION

The present invention relates to a cardiac myocyte protective agent and a cardiac disease therapeutic agent, comprising serofendic acid.

### BACKGROUND

Mitochondria are intracellular organelles having a main function of synthesizing ATP by oxidative phosphorylation, and play crucial roles in cell death in response to a variety of stresses such as myocardial ischemia/reperfusion⁽¹⁻³⁾. The opening of the mitochondrial permeability transition pore (MPTP), a non-specific pore that opens at the contact site between outer and inner mitochondrial membranes, results in the loss of mitochondrial membrane potential ΔΨₘ, matrix swelling, and the release of cytochrome c and other proapoptotic factors that lead to cell death⁽⁴⁻⁶⁾. Mitochondrial matrix calcium ([Ca²⁺]ₘ) overload and reactive oxygen species (ROS) favor MPTP opening⁽⁷⁾. Accordingly, inhibition of MPTP opening by preventing [Ca²⁺]ₘ overload and ROS generation will be an effective strategy for the protection of hearts from ischemia/reperfusion injury.

The present inventors have shown that ATP-sensitive potassium channels located in the inner mitochondrial membrane (mitoK_{ATP} channels) play a central role in the signaling cascade of protection against oxidative stress in cardiac ventricular myocytes^{(8,9)} and cerebellar granule neurons^{(10,11)}. MitoK_{ATP} channels prevent [Ca²⁺]ₘ overload and ROS generation, thereby inhibiting MPTP opening in both types of cells^{(10,12-14)}.

Diazoxide, a selective opener of mitoK_{ATP} channels, has been shown to have protective effects against myocardial ischemia/reperfusion both in vitro^{(15,16)} (and in vivo^{(17,18)}. Unfortunately, the clinical use of this agent has been hampered due to unwanted side effects, such as excessive hypotension or edema.

The present inventors previously purified a novel neuroprotective substance named ‘serofendic acid’ (SFA) derived from the lipophilic fraction of fetal calf serum⁽¹⁹⁾. This compound is a diterpenoid substance and is an endogenous substance having a low molecular weight of 382 Da. The chemical structure of serofendic acid has been checked by mass spectrometry and nuclear magnetic resonance (NMR) analysis, and serofendic acid has been found to be a sulfur-containing atisane-type diterpenoid (15-hydroxy-17-methylsulfinyl-atisan-19-acid), which is an epimer mixture having an inverted configuration in the sulfoxide group⁽²³⁾. A naturally occurring atisane derivative contained in plants has been reported, but serofendic acid is the first atisane derivative found in mammals. The compound exhibited the ability to protect cultured cortical and striatal neurons against glutamate, nitric oxide, and H₂O₂ cytotoxicity⁽¹⁹⁻²²⁾. Synthetic serofendic acid is known to exhibit a strong in vitro protective action on neurocytes against cytotoxicity of ROS.

### REFERENCE DOCUMENTS

1. Green D R, Kroemer G. The pathophysiology of mitochondrial cell death. Science. 2004; 305:626-9
2. Kroemer G, Dallaporta B, Resche-Rigon M. The mitochondrial death/life regulator in apoptosis and necrosis. Annu Rev Physiol. 1998; 60:619-42.
3. Weiss J N, Korge P, Honda H M, Ping P. Role of the mitochondrial permeability transition in myocardial disease. Circ Res. 2003; 93:292-301.
4. Crompton M. The mitochondrial permeability transition pore and its role in cell death. Biochem J. 1999; 341 (Pt 2):233-49.
5. Crow M T, Mani K, Nam Y J, Kitsis R N. The mitochondrial death pathway and cardiac myocyte apoptosis. Circ Res. 2004; 95:957-70.
6. Halestrap A P, Clarke S J, Javadov S A. Mitochondrial permeability transition pore opening during myocardial reperfusion—a target for cardioprotection. Cardiovasc Res. 2004; 61:372-85.
7. Brookes P S, Yoon Y, Robotham J L, Anders M W, Sheu S S. Calcium, ATP, and ROS: a mitochondrial love-hate triangle. Am J Physiol Cell Physiol. 2004; 287:C817-33.
8. Akao M, Ohler A, O'Rourke B, Marban E. Mitochondrial ATP-sensitive potassium channels inhibit apoptosis induced by oxidative stress in cardiac cells. Circ Res. 2001; 88:1267-75.
9. Akao M, Teshima Y, Marban E. Antiapoptotic effect of nicorandil mediated by mitochondrial ATP-sensitive potassium channels in cultured cardiac myocytes. J Am Coll Cardiol. 2002; 40:803-10.
10. Teshima Y, Akao M, Li R A, Chong T H, Baumgartner W A, Johnston M V, Marban E. Mitochondrial ATP-sensitive potassium channel activation protects cerebellar granule neurons from apoptosis induced by oxidative stress. Stroke. 2003; 34:1796-802.
11. Teshima Y, Akao M, Baumgartner W A, Marban E. Nicorandil prevents oxidative stress-induced apoptosis in neurons by activating mitochondrial ATP-sensitive potassium channels. Brain Res. 2003; 990:45-50.
12. Akao M, O'Rourke B, Kusuoka H, Teshima Y, Jones S P, Marban E. Differential actions of cardioprotective agents on the mitochondrial death pathway. Circ Res. 2003; 92:195-202.
13. Akao M, O'Rourke B, Teshima Y, Seharaseyon J, Marban E. Mechanistically distinct steps in the mitochondrial death pathway triggered by oxidative stress in cardiac myocytes. Circ Res. 2003; 92:186-94.
14. Murata M, Akao M, O'Rourke B, Marban E. Mitochondrial ATP-sensitive potassium channels attenuate matrix Ca²⁺ overload during simulated ischemia and reperfusion: possible mechanism of cardioprotection. Circ Res. 2001; 89:891-8.
15. Garlid K D, Paucek P, Yarov-Yarovoy V, Murray H N, Darbenzio R B, D'Alonzo A J, Lodge N J, Smith M A, Grover G J. Cardioprotective effect of diazoxide and its interaction with mitochondrial ATP-sensitive K⁺ channels. Possible mechanism of cardioprotection. Circ Res. 1997; 81:1072-82.
16. Liu Y, Sato T, O'Rourke B, Marban E. Mitochondrial ATP-dependent potassium channels: novel effectors of cardioprotection? Circulation. 1998; 97:2463-9.
17. Fryer R M, Eells J T, Hsu A K, Henry M M, Gross G J. Ischemic preconditioning in rats: role of mitochondrial K_{ATP} channel in preservation of mitochondrial function. Am J Physiol Heart Circ Physiol. 2000; 278:H305-12.
18. Miura T, Liu Y, Kita H, Ogawa T, Shimamoto K. Roles of mitochondrial ATP-sensitive K channels and PKC in anti-infarct tolerance afforded by adenosine A1 receptor activation. J Am Coll Cardiol. 2000; 35:238-45.
19. Kume T, Asai N, Nishikawa H, Mano N, Terauchi T, Taguchi R, Shirakawa H, Osakada F, Mori H, Asakawa N, Yonaga M, Nishizawa Y, Sugimoto H, Shimohama S, Katsuki H, Kaneko S, Akaike A. Isolation of a diterpenoid substance with potent neuroprotective activity from fetal calf serum. Proc Natl Acad Sci USA. 2002; 99:3288-93.
20. Akaike A, Katsuki H, Kume T. Pharmacological and physiological properties of serofendic acid, a novel neuroprotective substance isolated from fetal calf serum. Life Sci. 2003; 74:263-9.
21. Taguchi R, Nishikawa H, Kume T, Terauchi T, Kaneko S, Katsuki H, Yonaga M, Sugimoto H, Akaike A. Serofendic acid prevents acute glutamate neurotoxicity in cultured cortical neurons. Eur J. Pharmacol. 2003; 477:195-203.
22. Osakada F, Kawato Y, Kume T, Katsuki H, Sugimoto H, Akaike A. Serofendic acid, a sulfur-containing diterpenoid derived from fetal calf serum, attenuates reactive oxygen species-induced oxidative stress in cultured striatal neurons. J Pharmacol Exp Ther. 2004; 311:51-9.
23. Terauchi, T., Asai, N., Yonaga, M., Mano, N., Kume, T., Akaike, A., Sugimoto, H., Synthesis and absolute configuration of serofendic acids. Tetrahedron Lett. 43, 3625-3628, 2002

### DETAILED DESCRIPTION

The present invention has an object of providing a cardiac myocyte protective agent and a cardiac disease therapeutic agent, comprising an atisane-type diterpene compound.

As a result of conducting active studies for attaining the above-described object, the present inventors found that serofendic acid has a protective effect against heart function failure and injury caused by ischemia/reperfusion, and thus completed the present invention. Namely, the present invention is as follows.

(1) A cardiac myocyte protective agent, comprising a compound represented by the formula (Ia) below, a pharmaceutically acceptable salt thereof, or a solvate thereof:

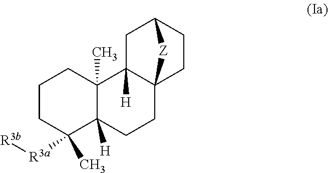

wherein:

Z refers to a group represented by the formula below:

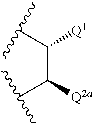

wherein:
Q¹ refers to the formula represented by the formula:

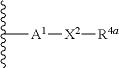

wherein:
   A¹ refers to an optionally substituted C₁₋₆ alkylene group or a single bond;
   X² refers to the formula —S(O)ₘ—, wherein m refers to an integer of 0, 1, or 2, an oxygen atom, a carbonyl group, or a single bond; and
   R^{4a} refers to a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
Q^{2a} refers to a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, or a group represented by the formula —NR^{6a}R^{6b}, wherein R^{6a} and R^{6b} are each independently a hydrogen atom, an optionally substituted C₁₋₆ alkyl group, an optionally substituted C₂₋₆ alkenyl group, an optionally substituted C₂₋₆ alkynyl group, an optionally substituted C₂₋₇ acyl group, or an optionally substituted C₁₋₆ alkylsulfonyl group;

R^{3a} refers to a carbonyl group, a methylene group or a single bond; and

R^{3b} refers to a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, a cyano group, or a group represented by the formula —NR^{5a}R^{5b}, wherein R^{5a} and R^{5b} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group.

(2) In the compound represented by the formula (Ia) above, R^{3a} may be, for example, a carbonyl group; R^{3b} may be, for example, a C₁₋₆ alkoxy group or a hydroxyl group; and Q^{2a} may be, for example, a C₁₋₆ alkoxy group or a hydroxyl group.

Also according to the present invention, Q¹ is preferably a group represented by the formula -A¹-S(O)ₘ—R^{4a}, wherein:

A¹ refers to an optionally substituted C₁₋₆ alkylene group or a single bond;

R^{4a} refers to a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and

m refers to an integer of 0, 1, or 2. Among the above, R^{4a} is preferably an optionally substituted methyl group, an optionally substituted ethyl group, an optionally substituted n-propyl group, or an optionally substituted i-propyl group.

In the formula (Ia) above, m is, for example, 1; and A¹ is, for example, a methylene group.

Also according to the present invention, A¹ may be a C₁₋₆ alkylene group or a single bond; and R^{4a} may be a hydrogen atom, a C₁₋₆ alkyl group, a C₂₋₆ alkenyl group, a C₂₋₆ alkynyl group, a C₆₋₁₄ aromatic hydrocarbon cyclic group, a 5- to 14-membered aromatic heterocyclic group, a C₁₋₆ alkoxy group, or a hydroxyl group.

Also according to the present invention, Q^{2a} may be, for example, a hydrogen atom, a halogen atom, a C₁₋₆ alkyl group, a C₂₋₆ alkenyl group, a C₂₋₆ alkynyl group, a C₁₋₆ alkoxy group, a hydroxyl group, or a group represented by the formula —NR^{6a}R^{6b}, wherein R^{6a} and R^{6b} are each independently a hydrogen atom, a C₁₋₆ alkyl group, a C₂₋₆ alkenyl group, a C₂₋₆ alkynyl group, a C₂₋₇ acyl group, or a C₁₋₆ alkylsulfonyl group.

The above protective agent can be used to protect cardiac myocytes against cardiac ischemia/reperfusion injury, and also to protect cardiac myocytes against cardiac myocyte disorders caused by reactive oxygen species (e.g., hydroxy radicals).

The compound represented by the formula (Ia) above may be, for example, any one selected from the group consisting of serofendic acid, ent-15β-hydroxy-17-methylthio-16α-atisan-19-oic acid, methyl ent-15β-hydroxy-17-methylsulfinyl-16α-atisan-19-oate, ent-15β-hydroxy-17-methylsulfonyl-16α-atisan-19-oic acid, ent-15α-hydroxy-17-methylsulfinyl-16β-atisan-19-oic acid, ent-15β,19-dihydroxy-17-methylsulfinyl-16α-atisane, ent-15β-hydroxy-17-methylsulfinyl-16α-atisane, ent-17-methylsulfinyl-15-oxo-16α-atisan-19-oic acid, ent-15β-hydroxy-17-propylsulfinyl-16α-atisan-19-oic acid, and ent-17-acetyl-15β-hydroxy-16α-atisan-19-oic acid. In the present invention, serofendic acid is preferable.

(3) The present invention is also directed to a cardiac disease prophylactic and/or therapeutic agent, comprising a compound represented by the formula (Ia) shown in (1), a pharmaceutically acceptable salt thereof, or a solvate thereof. The compound represented by the formula (Ia) above may be, for example, any one selected from the group consisting of serofendic acid, ent-15β-hydroxy-17-methylthio-16α-atisan-19-oic acid, methyl ent-15β-hydroxy-17-methylsulfinyl-16α-atisan-19-oate, ent-15β-hydroxy-17-methylsulfonyl-16α-atisan-19-oic acid, ent-15α-hydroxy-17-methylsulfinyl-16β-atisan-19-oic acid, ent-15β,19-dihydroxy-17-methylsulfinyl-16α-atisane, ent-15β-hydroxy-17-methylsulfinyl-16α-atisane, ent-17-methylsulfinyl-15-oxo-16α-atisan-19-oic acid, ent-1513-hydroxy-17-propylsulfinyl-16α-atisan-19-oic acid, and ent-17-acetyl-15β-hydroxy-16α-atisan-19-oic acid. In the present invention, serofendic acid is preferable.
(4) The present invention is also directed to a cardiac ischemia/reperfusion injury prophylactic and/or therapeutic agent, comprising a compound represented by the formula (Ia) shown in (1), a pharmaceutically acceptable salt thereof, or a solvate thereof. The compound represented by the formula (Ia) above may be, for example, any one selected from the group consisting of serofendic acid, ent-15β-hydroxy-17-methylthio-16α-atisan-19-oic acid, methyl ent-15β-hydroxy-17-methylsulfinyl-16α-atisan-19-oate, ent-15β-hydroxy-17-methylsulfonyl-16α-atisan-19-oic acid, ent-15α-hydroxy-17-methylsulfinyl-1613-atisan-19-oic acid, ent-15β,19-dihydroxy-17-methylsulfinyl-16α-atisane, ent-15β-hydroxy-17-methylsulfinyl-16α-atisane, ent-17-methylsulfinyl-15-oxo-16α-atisan-19-oic acid, ent-15β-hydroxy-17-propylsulfinyl-16α-atisan-19-oic acid, and ent-17-acetyl-15β-hydroxy-16α-atisan-19-oic acid. In the present invention, serofendic acid is preferable.

### BRIEF DESCRIPTION OF THE DRAWINGS

FIG. 1 shows TUNEL staining and DAPI staining, and results of MTS assay.

FIG. 2 shows the mitochondrial inner membrane potential (ΔΨₘ) in neonatal rat cardiac myocytes.

FIG. 3 shows the results of time-lapse analysis of ΔΨₘ loss in neonatal rat cardiac myocytes.

FIG. 4 shows the results of time-lapse analysis of intracellular ROS production in neonatal rat cardiac myocytes.

FIG. 5 shows the results of time-lapse analysis of intracellular [Ca²⁺]ₘ in neonatal rat cardiac myocytes.

FIG. 6 shows a time-lapse change in the blood pressure of SD-line male rats.

Hereinafter, the present invention will be described in detail. The following embodiments are illustrative of the present invention, and it is not intended to limit the present invention to the following embodiments. The present invention may be carried out in various forms without departing from the gist thereof.

The documents, laid-open patent publications, patents and other patent documents referred to in this specification are incorporated herein by reference. This specification encompasses the contents of the specification of the Japanese patent application, filed on Sep. 8, 2005, upon which the present application claims the benefit of priority (Japanese Patent Application No. 2005-260205).

The present invention is directed to a cardiac myocyte protective agent, and a prophylactic and/or therapeutic agent of ischemia/reperfusion injury and cardiac diseases, comprising a compound represented by the formula (Ia), a pharmaceutically acceptable salt thereof, or a solvate thereof (for example, serofendic acid (SFA)).

The present inventors considered that a compound represented by the formula (Ia) (for example, SFA) has a cardioprotective effect against myocardial ischemia/reperfusion injury, and examined whether or not SFA has a protective effect on cardiac myocytes of neonatal rats against oxidative stress. As a result, the present inventors obtained the findings that pretreatment with SFA significantly suppressed markers of cell death, as assessed by TUNEL staining and cell viability assay, at 16 hours after H₂O₂ treatment to induce cell death, in primary cultures of neonatal rat cardiac myocytes exposed to oxidative stress

Loss of mitochondrial membrane potential (ΔΨₘ) is a critical step of the death pathway, which is triggered by matrix calcium overload and reactive oxygen species. SFA prevented the ΔΨₘ loss induced by H₂O₂ in a concentration-dependent manner. SFA remarkably suppressed the H₂O₂-induced matrix calcium overload and intracellular accumulation of reactive oxygen species. The present inventors found that the protective effect of SFA was comparable with that of a mitochondrial ATP-sensitive potassium (mitoK_{ATP}) channel opener, diazoxide. Diazoxide causes side effects such as blood pressure drop, edema, and the like, whereas SFA does not act on vascular smooth muscle and thus does not cause blood pressure drop. Therefore, SFA may be considered to be useful as a novel cardiac myocyte protective agent.

Furthermore, the mitoK_{ATP} channel blocker, 5-hydroxydecanoate (5-HD), abolished the protective effect of SFA. Co-application of SFA and diazoxide did not show an additive effect. Thus, SFA inhibited the oxidant-induced mitochondrial death pathway, presumably through activation of the mitoK_{ATP} channel.

The present invention has been completed based on the above-described findings.

### 1. Compounds Usable in the Present Invention

Examples of compounds usable in the present invention include a compound represented by the formula (Ia) below, a pharmaceutically acceptable salt thereof, or a solvate thereof.

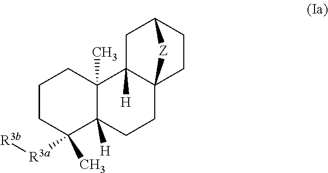

wherein:

Z refers to a group represented by the formula below:

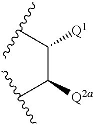

wherein:
Q¹ refers to the formula represented by the formula:

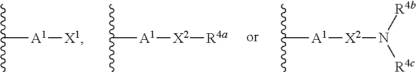

wherein:
   A¹ refers to an optionally substituted C₁₋₆ alkylene group or a single bond;
   X¹ refers to a halogen atom or a cyano group;
   X² refers to the formula —S(O)ₘ—, wherein m refers to an integer of 0, 1, or 2, an oxygen atom, a carbonyl group, or a single bond;
   R^{4a} refers to a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
   R^{4b} and R^{4c} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group; and
Q^{2a} refers to a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, or a group represented by the formula —NR^{6a}R^{6b}, wherein R^{6a} and R^{6b} are each independently a hydrogen atom, an optionally substituted C₁₋₆ alkyl group, an optionally substituted C₂₋₆ alkenyl group, an optionally substituted C₂₋₆ alkynyl group, an optionally substituted C₂₋₇ acyl group, or an optionally substituted C₁₋₆ alkylsulfonyl group;

R^{3a} refers to a carbonyl group, a methylene group or a single bond; and

R^{3b} refers to a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, a cyano group, or a group represented by the formula —NR^{5a}R^{5b}, wherein R^{5a} and R^{5b} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group.

Herein, a chemical formula may occasionally represent a certain isomer for the sake of convenience, but the present invention encompasses all the geometrical isomers, asymmetric carbon-based optical isomers, stereoisomers, tautomers, and other isomers and isomer mixtures which can occur based on the structure of the compound. The present invention is not limited to any formula provided for the sake of convenience, and a compound of the present invention may be one of the isomers or a mixture thereof. Accordingly, a molecule may occasionally have an asymmetric carbon atom and thus may have an optical activator or a racemic body, but the present invention encompasses any of such forms with no specific limitation. A molecule may occasionally have a polymorphism, but a compound of the present invention may be any single crystal form or a mixture of crystal forms with no specific limitation. A compound of the present invention may be either an anhydride or a hydrate.

Herein, the “C₁₋₆ alkyl group” refers to a straight-chain or branched-chain alkyl group having a carbon number of 1 to 6. Specific examples of such a group include methyl group, ethyl group, n-propyl group, i-propyl group, n-butyl group, i-butyl group, tert-butyl group, n-pentyl group, i-pentyl group, neopentyl group, n-hexyl group, 1-methylpropyl group, 1,2-dimethylpropyl group, 2-ethylpropyl group, 1-methyl-2-ethylpropyl group, 1-ethyl-2-methylpropyl group, 1,1,2-trimethylpropyl group, 1-methylbutyl group, 2-methylbutyl group, 1,1-dimethylbutyl group, 2,2-dimethylbutyl group, 2-ethylbutyl group, 1,3-dimethylbutyl group, 2-methylpentyl group, 3-methylpentyl group, and the like.

Herein, the “C₁₋₆ alkylene group” refers to a divalent group derived from the above-defined “C₁₋₆ alkyl group” with one hydrogen atom being deleted. Specific examples of such a group include methylene group, ethylene group, methylethylene group, propylene group, ethylethylene group, 1,1-dimethylethylene group, 1,2-dimethylethylene group, trimethylene group, 1-methyltrimethylene group, 1-ethyltrimethylene group, 2-methyltrimethylene group, 1,1-dimethyltrimethylene group, tetramethylene group, pentamethylene group, hexamethylene group, and the like; preferably methylene group, 1,2-ethylene group, 1,3-propylene group, and the like; and more preferably methylene group.

Herein, the “C₂₋₆ alkenyl group” refers to a straight-chain or branched-chain alkenyl group having a carbon number of 2 to 6. Specific examples of such a group include vinyl group, allyl group, 1-propenyl group, isopropenyl group, 1-butene-1-yl group, 1-butene-2-yl group, 1-butene-3-yl group, 2-butene-1-yl group, 2-butene-2-yl group, and the like.

Herein, the “C₂₋₆ alkynyl group” refers to a straight-chain or branched-chain alkynyl group having a carbon number of 2 to 6. Specific examples of such a group include ethynyl group, 1-propynyl group, 2-propynyl group, butynyl group, pentynyl group, hexynyl group, and the like.

Herein, the “C₆₋₁₄ aromatic hydrocarbon cyclic group” refers to an aromatic cyclic group having a carbon number of 6 to 14. Specific examples of such a group include phenyl group, 1-naphthyl group, 2-naphthyl group, as-indacenyl group, s-indacenyl group, acenaphthylenyl group, and the like; and preferably phenyl group, 1-naphthyl group, and 2-naphthyl group.

Herein, the “5- to 14-membered aromatic heterocyclic group” refers to an aromatic cyclic group in which the number of atoms forming the cycle of the cyclic group is 5 to 14 and the type of the atoms forming the cycle of the cyclic group is carbon atom or hetero atom. Specific examples of such a group include pyridine, thiophene, furan, pyrrole, oxazole, isoxazole, thiazole, isothiazole, imidazole, triazole, pyrazole, furazan, thiadiazole, oxadiazole, pyridazine, pyrimidine, pyrazine, indole, isoindole, indazole, chromene, quinoline, isoquinoline, cinnoline, quinazoline, quinoxaline, naphthylidine, phthalazine, purine, pteridine, thienofuran, imidazothiazole, benzofuran, benzothiophene, benzoxazole, benzthiazole, benzthiadiazole, benzimidazole, imidazopyridine, pyrrolopyridine, pyrrolopyrimidine, pyridopyrimidine, and the like.

Herein, the “C₃₋₈ cycloalkyl group” refers to a cyclic aliphatic hydrocarbon group having a carbon number of 3 to 8. Specific examples of such a group include cyclopropyl group, cyclobutyl group, cyclopentyl group, cyclohexyl group, cycloheptyl group, cyclopropenyl group, cyclobutenyl group, cyclopentenyl group, cyclohexenyl group, cycloheptenyl group, and the like.

Herein, the “halogen atom” refers to fluorine atom, chlorine atom, bromine atom, and iodine atom.

Herein, the “C₁₋₆ alkoxy group” refers to an oxy group having the above-defined “C₁₋₆ alkyl group” bonded thereto. Specific examples of such a group include methoxy group, ethoxy group, n-propoxy group, i-propoxy group, n-butoxy group, i-butoxy group, sec-butoxy group, t-butoxy group, n-pentyloxy group, i-pentyloxy group, sec-pentyloxy group, t-pentyloxy group, neopentyloxy group, 1-methylbutoxy group, 2-methylbutoxy group, 1,1-dimethylpropoxy group, 1,2-dimethylpropoxy group, n-hexyloxy group, i-hexyloxy group, 1-methylpentyloxy group, 2-methylpentyloxy group, 3-methylpentyloxy group, 1,1-dimethylbutoxy group, 1,2-dimethylbutoxy group, 2,2-dimethylbutoxy group, 1,3-dimethylbutoxy group, 2,3-dimethylbutoxy group, 3,3-dimethylbutoxy group, 1-ethylbutoxy group, 2-ethylbutoxy group, 1,1,2-trimethylpropoxy group, 1,2,2-trimethylpropoxy group, 1-ethyl-1-methylpropoxy group, 1-ethyl-2-methylpropoxy group, and the like.

Herein, the “C₂₋₇ acyl group” refers to a carbonyl group having the above-defined “C₁₋₆ alkyl group” bonded thereto. Specific examples of such a group include acetyl group, propionyl group, butylyl group, isobutylyl group, and the like.

Herein, the “C₁₋₆ alkoxycarbonyl group” refers to a carbonyl group having the above-defined “C₁₋₆ alkoxy group” bonded thereto. Specific examples of such a group include methoxycarbonyl group, ethoxycarbonyl group, n-propoxycarbonyl group, i-propoxycarbonyl group, n-butoxycarbonyl group, i-butoxycarbonyl group, sec-butoxycarbonyl group, t-butoxycarbonyl group, and the like.

Herein, the “C₁₋₆ alkoxysulfonyl group” refers to a sulfonyl group having the above-defined “C₁₋₆ alkyl group” bonded thereto. Specific examples of such a group include methoxysulfonyl group, propylsulfonyl group, and the like.

Herein, “optionally substituted” is synonymous with “may have one or a plurality of substituents with an arbitrary combination at any substitutable position”. Specific examples of the substituent include:

(1) halogen atom,
(2) hydroxyl group,
(3) thiol group,
(4) nitro group,
(5) nitrile group,
(6) oxo group,
(7) azide group,
(8) guanidino group,
(9) hydrazino group,
(10) isocyano group,
(11) cyanate group,
(12) isocyanate group,
(13) thiocyanate group,
(14) isothiocyanate group,
(15) nitroso group,
(16) carbamide group,
(17) formyl group,
(18) C₁₋₆ imidoyl group,
(19) C₁₋₆ alkyl group, C₂₋₆ alkenyl group, C₂₋₆ alkynyl group, C₃₋₈ cycloalkyl group, C₁₋₆ alkoxy group, C₂₋₆ alkenyloxy group, C₂₋₆ alkynyloxy group, C₃₋₆ cycloalkyloxy group, C₁₋₆ alkylthio group, C₂₋₆ alkenylthio group, C₂₋₆ alkynylthio group, C₃₋₆ cycloalkylthio group, or C₁₋₆ alkylenedioxy group, each of which may be substituted with 1 to 3 halogen atom(s) or hydroxyl group(s),
(20) C₆₋₁₄ aryl group,
(21) 5- to 14-membered heterocyclic group,
(22) carboxyl group,
(23) trifluoromethyl group,
(24) C₆₋₁₄ aryl C₁₋₆ alkyl group,
(25) 5- to 14-membered heterocyclic C₁₋₆ alkyl group,
(26) C₁₋₆ alkylcarbamoyl group,
(27) C₁₋₆ alkoxycarbamoyl group,
(28) C₁₋₆ alkylcarbonyl group,
(29) C₁₋₆ alkylcarbonyloxy group,
(30) C₁₋₆ alkylsulfonyl group,
(31) C₁₋₆ alkylsulfinyl group, and the like.
Preferable examples among these substituent include:
(1) halogen atom,
(2) hydroxyl group,
(5) nitrile group,
(16) carbamide group,
(19) C₁₋₆ alkyl group, C₂₋₆ alkenyl group, C₂₋₆ alkynyl group, C₃₋₈ cycloalkyl group, C₁₋₆ alkoxy group, or C₃₋₆ cycloalkyloxy group, each of which may be substituted with 1 to 3 halogen atom(s) or hydroxyl group(s),
(20) C₆₋₁₄ aryl group,
(21) 5- to 14-membered heterocyclic group,
(22) carboxyl group,
(23) trifluoromethyl group,
(24) C₆₋₁₄ aryl C₁₋₆ alkyl group,
(25) 5- to 14-membered heterocyclic C₁₋₆ alkyl group,
(26) C₁₋₆ alkylcarbamoyl group,
(27) C₁₋₆ alkoxycarbamoyl group,
(28) C₁₋₆ alkylcarbonyl group,
(29) C₁₋₆ alkylcarbonyloxy group,
(30) C₁₋₆ alkylsulfonyl group,
(31) C₁₋₆ alkylsulfinyl group, and the like.
More preferable examples of the substituent include:
(1a) halogen atom,
(2a) hydroxyl group,
(3a) nitrile group,
(4a) C₁₋₆ alkyl group,
(5a) C₃₋₈ cycloalkyl group,
(6a) C₁₋₆ alkoxy group,
(7a) phenyl group,
(8a) carboxyl group,
(9a) C₁₋₆ alkylcarbonyl group,
(10a) C₁₋₆ alkylsulfinyl group, and the like.

Z refers to a divalent organic group comprising 2 to 3 carbon atoms, with proviso that Z has two groups selected from the groups represented by the formula:

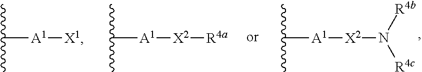

or a combination thereof, wherein A¹, X¹, X², R^{4a}, R^{4b}, and R^{4c} each have the same significance as above.

The “divalent organic group comprising 2 to 3 carbon atoms” refers to a divalent organic group which may optionally contain one oxygen atom as a carbonyl group, has a methylene group, a methine group, a carbon atom, or a carbonyl group as an atom involved in the structure of the cycle in Z, and is comprising 2 to 3 carbon atoms. Specific examples of such a group are represented by the formula:

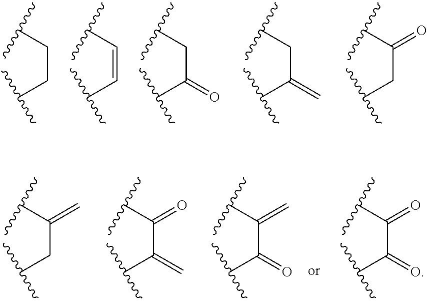

Examples of the “divalent organic group comprising 2 to 3 carbon atoms” preferably include 1,2-ethylene group, 1,2vinylene group, ethanone-1,2-ylene group, or 1-propene-2,3-ylene group, and the like; more preferably a group represented by the formula:

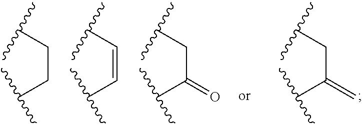

still more preferably a group represented by the formula:

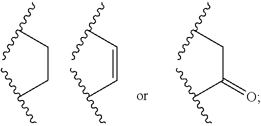

and most preferably a group represented by the formula:

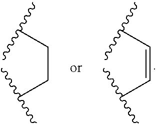

The formula:

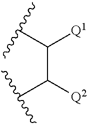

wherein Q¹ and Q² have the same significance as above, refers to a group including, at an arbitrary ratio, 1 to 4 group(s) selected from the group consisting of:

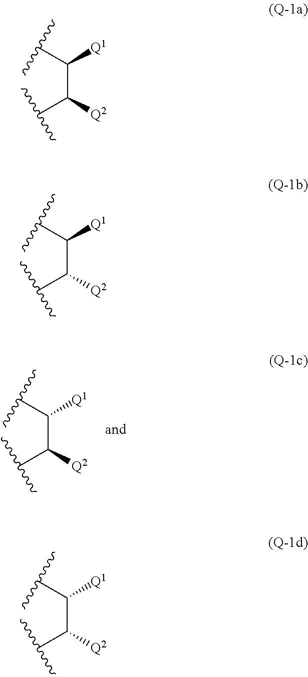

wherein Q¹ and Q² have the same significance as above; and preferably a group represented by (Q-1a), (Q-1b), (Q-1c) or (Q-1d) above.

The formula:

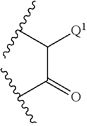

wherein Q¹ has the same significance as above, refers to a group including, at an arbitrary ratio, 1 to 2 group(s) selected from the group consisting of:

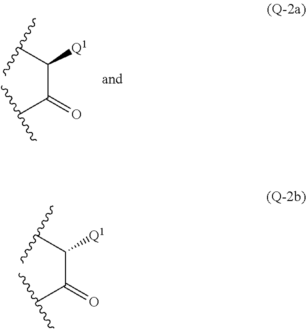

wherein Q¹ has the same significance as above; preferably a group represented by (Q-2a) or (Q-2b) above; more preferably a group represented by (Q-2a) above; and still more preferably a group represented by (Q-2b) above.

The formula:

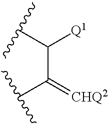

wherein Q¹ and Q² have the same significance as above, refers to a group including, at an arbitrary ratio, 1 to 2 group(s) selected from the group consisting of:

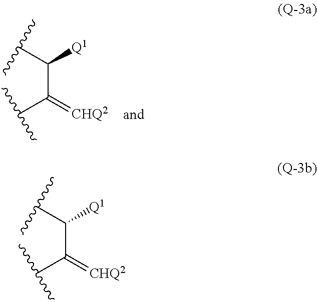

wherein Q¹ and Q² each have the same significance as above; preferably a group represented by (Q-3a) or (Q-3b) above; more preferably a group represented by (Q-3a) above; and still more preferably a group represented by (Q-3b) above.

In the case where Z in the compound (Ia) is a group represented by the formula:

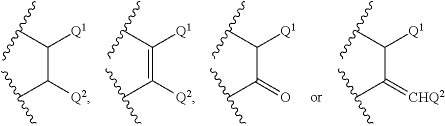

wherein Q¹ and Q² have the same significance as above, the compound (Ia) is a group represented by the formula:

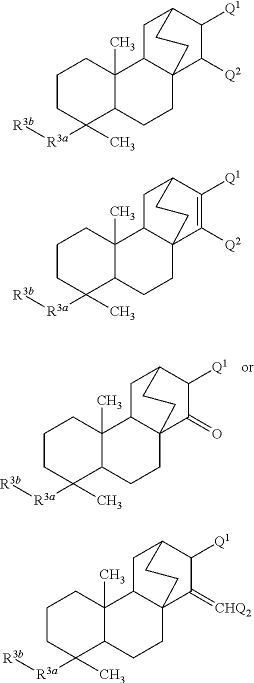

wherein Q¹, Q², R^{3a} and R^{3b} have the same significance as above.

Z preferably refers to a group represented by the formula:

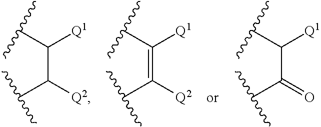

wherein Q¹ and Q² have the same significance as above; more preferably refers to a group represented by the formula:

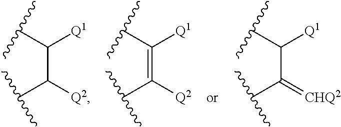

wherein Q¹ and Q² have the same significance as above; still more preferably refers to a group represented by the formula:

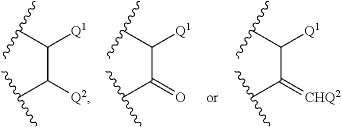

wherein Q¹ and Q² have the same significance as above; and most preferably refers to a group represented by the formula:

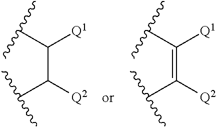

wherein Q¹ and Q² have the same significance as above.

The formula —R^{3a}—R^{3b} refers to a group defined above; preferably refers to a carboxyl group, a C₁₋₆ alkoxycarbonyl group, a C₁₋₆ alkyl group, a cyano group, or a group represented by the formula —CO—NR^{5a}R^{5b} (wherein R^{5a} and R^{5b} have the same significance as above) or the formula —CH₂—R^{30b} (wherein R^{30b} refers to a C₁₋₆ alkoxy group or a halogen atom); more preferably may refer to a carboxyl group, a C₁₋₆ alkoxycarbonyl group, a C₁₋₆ alkyl group, a cyano group, or a group represented by the formula —CO—NR^{5a}R^{5b} (wherein R^{5a} and R^{5b} have the same significance as above); still more preferably may refer to a carboxyl group, a methoxycarbonyl group, a methyl group or a cyano group; and most preferably may refer to a carboxyl group.

Q² refers to a group defined above; preferably refers to a hydroxyl group, a C₁₋₆ alkoxy group, a halogen atom, or a group represented by the formula —NR^{4b}R^{4c} (wherein R^{4b} and R^{4b} have the same significance as above); more preferably may refer to a hydroxyl group, a halogen atom, or —NH₂; and most preferably refers to a hydroxyl group.

Q^{2a} refers to a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, or a group represented by the formula —NR^{6a}R^{6b} (wherein R^{6a} and R^{6b} are each independently a hydrogen atom, an optionally substituted C₁₋₆ alkyl group, an optionally substituted C₂₋₆ alkenyl group, an optionally substituted C₂₋₆ alkynyl group, an optionally substituted C₂₋₇ acyl group, or an optionally substituted C₁₋₆ alkylsulfonyl group).

Q^{2a} preferably refers to a halogen atom, an optionally substituted C₁₋₆ alkyl group, an optionally substituted C₁₋₆ alkoxy group, a hydroxyl group, or a group represented by the formula —NR^{6a}R^{6b} (wherein R^{6a} and R^{6b} are each independently a hydrogen atom, an optionally substituted C₁₋₆ alkyl group, an optionally substituted C₂₋₇ acyl group, or an optionally substituted C₁₋₆ alkylsulfonyl group); more preferably refers to a halogen atom, a C₁₋₆ alkoxy group, a hydroxyl group, or —NH₂; and still more preferably refers to a hydroxyl group.

Q^{2b} refers to a hydrogen atom, or an optionally substituted C₁₋₆ alkyl group; preferably refers to a hydrogen atom or a methyl group; and more preferably refers to a hydrogen atom.

Q¹ refers to a group represented by the formula -A¹⁰-X¹⁰ (wherein A¹⁰ refers to an optionally substituted C₁₋₆ alkylene group or a single bond, and X¹⁰ refers to a group represented by the formula —X¹, X²—R^{4a} or —X²—NR^{4b}R^{4c} (wherein X¹, X², R^{4a}, R^{4b} and R^{4c} have the same significance as above)).

A¹⁰ may preferably refer to a C₁₋₆ alkylene group and a single bond; more preferably a group represented by the formula —(CH₂)ₛ— (wherein s refers to an integer of 0 to 6); and still more preferably a single bond, a methylene group, or an ethylene group; and most preferably a methylene group.

X¹⁰ preferably refers to a halogen atom, a cyano group, or a group represented by the formula:

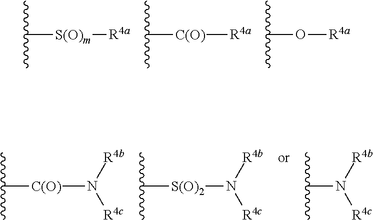

wherein m, R^{4a}, R^{4b}, and R^{4c} have the same significance as above; more preferably refers to a group represented by the formula:

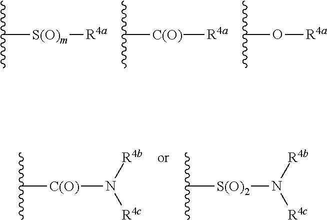

wherein m, R^{4a}, R^{4b}, and R^{4c} have the same significance as above; and still more preferably refers to a group represented by the formula —S(O)—R^{4a}, wherein R^{4a} has the same significance as above.

R^{4a} refers to a group defined above; preferably a hydrogen atom, an optionally substituted C₁₋₆ alkyl group, an optionally substituted C₆₋₁₄ aromatic hydrocarbon cyclic group, or an optionally substituted 5- to 14-membered aromatic heterocyclic group; more preferably a hydrogen atom, a C₁₋₆ alkyl group, or a C₆₋₁₄ aromatic hydrocarbon cyclic group; still more preferably a methyl group, an ethyl group, an n-propyl group, or an i-propyl group; and most preferably a methyl group.

R^{4b} and R^{4c} refer to a group defined above; preferably a hydrogen atom, an optionally substituted C₁₋₆ alkyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group; more preferably a hydrogen atom, a C₁₋₆ alkyl group, a C₆₋₁₄ aromatic hydrocarbon cyclic group, a C₂₋₇ acyl group, or a C₁₋₆ alkylsulfonyl group; still more preferably a hydrogen atom, a C₁₋₆ alkyl group, or a C₁₋₆ alkylsulfonyl group; and most preferably a hydrogen atom, a methyl group, or an ethyl group.

A compound represented by the formula (Ia) can be chemically synthesized by a method described in International PCT Application Publication WO 02/088061 (e.g., any method described in “production method A” to “production method G” or any method described in the examples).

According to the present invention, it is preferable to use, among the compounds represented by the formula (Ia), serofendic acid (formula I) or any one selected from the compounds represented by formulas (II) to (X). The structural formulas of serofendic acid (formula I) and the compounds represented by formulas (II) to (X) will be shown below.

### Serofendic Acid

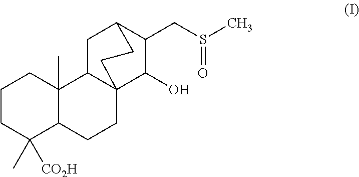

### ent-15β-hydroxy-17-methylthio-16α-atisan-19-oic acid

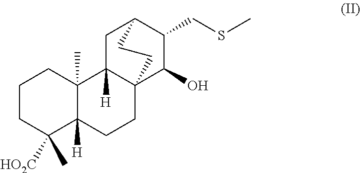

### methyl ent-15β-hydroxy-17-methylsulfinyl-16α-atisan-19-oate

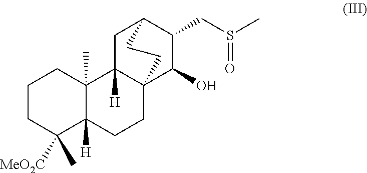

### ent-15β-hydroxy-17-methylsulfonyl-16α-atisan-19-oic acid

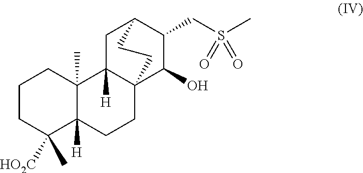

### ent-15α-hydroxy-17-methylsulfinyl-16β-atisan-19-oic acid

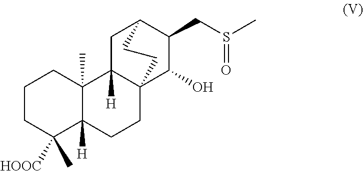

### ent-15β,19-dihydroxy-17-methylsulfinyl-16α-atisane

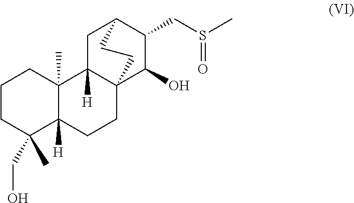

### ent-15β-hydroxy-17-methylsulfinyl-16α-atisane

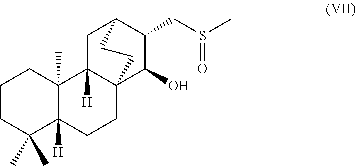

### ent-17-methylsulfinyl-15-oxo-16α-atisan-19-oic acid

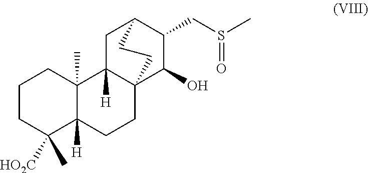

### ent-15β-hydroxy-17-propylsulfinyl-16α-atisan-19-oic acid

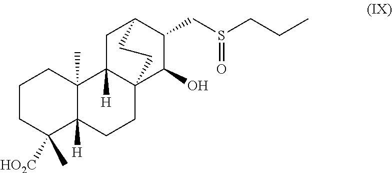

### ent-17-acetyl-15β-hydroxy-16α-atisan-19-oic acid

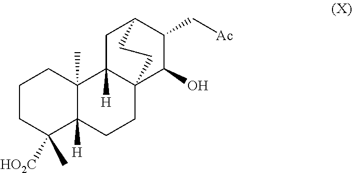

The compounds represented by the formulas (II) to (X) can be chemically synthesized by a method described in International PCT Application Publication WO 02/088061 (by methods described in examples 5, 8, 9, 12, 22, 38, 56, 63, and 100, respectively).

### 2. Serofendic Acid

According to the present invention, serofendic acid is preferably used among the above-identified compounds. Serofendic acid exerts remarkable protective action in vitro on cultured cerebral cortex neurons against glutamic acid neurotoxicity and NO neurotoxicity. Serofendic acid does not directly react with NO radicals, but suppresses the generation of hydroxy radicals (OH.) which are known to mediate cytotoxicity in the cascade of NO neurotoxicity. Namely, serofendic acid may be considered to be a low molecular weight physiologically active substance for promoting the survival of neurons in the central nervous system by reducing and weakening free radical-induced disorders. Serofendic acid also suppresses the generation of hydroxy radicals, which is the most reactive molecule among reactive oxygen species, and therefore is expected to act on the intractable diseases or inflammatory diseases in which cellular disorders caused by the active oxygen is involved. The present invention has clarified the protective action of serofendic acid on cardiac myocyte disorders caused by active oxygen.

The serofendic acid according to the present invention is a diterpenoid substance having a molecular weight of 382 which is represented by the formula (I) below.

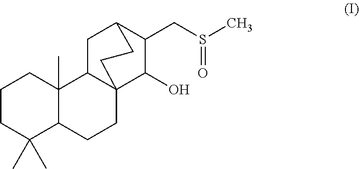

Based on the mass spectrometry and NMR analysis, serofendic acid has been found to be an epimer mixture which contains a sulfur molecule in the chemical structure thereof, has a cyclic diterpene called atisane as the basic framework, has a dimethylsulfoxide group, a carboxyl group or the like in the side chain, and has an inverted configuration in the sulfoxide group (International PCT Application Publication WO 02/088061 or Terauchi, T., Asai, N., Yonaga, M., Mano, N., Kume, T., Akaike, A., Sugimoto, H., Synthesis and absolute configuration of serofendic acids. Tetrahedron Lett. 43, 3625-3628, 2002). The chemical structure of serofendic acid (ent-15β-hydroxy-17-methylsulfinyl-16α-atisan-19-oic acid or 15-hydroxy-17-methylsulfinylatisan-19-oic acid) is very unique with no similarity to those of conventionally known substances. According to the present invention, such an atisane-type diterpene having a sulfoxide group can be used as a cardiac myocyte protective agent and a cardiac disease therapeutic agent.

According to the present invention, serofendic acid may be extracted from an in vivo sample (e.g., serum) or chemically synthesized. Serofendic acid can be extracted from serum by, for example, the method of Kume, T. et al. (Proc Natl Acad Sci USA. 2002; 99: 3288-93). Serofendic acid can be chemically synthesized by a method described in International PCT Application Publication WO 02/088061 or Terauchi, T., Asai, N., Yonaga, M., Mano, N., Kume, T., Akaike, A., Sugimoto, H., Synthesis and absolute configuration of serofendic acids. Tetrahedron Lett. 43, 3625-3628, 2002, using known compounds or the like as the materials.

### 3. Pharmaceutically Acceptable Salt and Solvate

The present invention encompasses use of any pharmaceutically acceptable salt of a compound represented by the formula (Ia) (e.g., serofendic acid). There is no specific limitation on the “pharmaceutically acceptable salt”, and examples thereof include hydrohalogenate (e.g., hydrofluoride, hydrochloride, hydrobromide, hydroiodide, etc.), inorganic acid salt (e.g., sulfate, nitrate, perchlorate, phosphate, carbonate, bicarbonate, etc.), organic carboxylate (e.g., acetate, oxalate, maleate, tartrate, fumarate, citrate, etc.), organic sulfonate (e.g., methanesulfonate, trifluoromethanesulfonate, ethanesulfonate, benzenesulfonate, toluenesulfonate, camphorsulfonate, etc.), amino acid salt (e.g., aspartate, glutamate, etc.), quaternary amine salt, alkaline metal salt (e.g., sodium salt, potassium salt, etc.), alkaline-earth metal salt (e.g., magnesium salt, calcium salt, etc.), and the like.

According to the present invention, a compound represented by the formula (Ia) or a pharmaceutically acceptable salt thereof may be an anhydride or may form a solvate such as a hydrate. The solvate may be either a hydrate or a non-hydrate, but is preferably a hydrate. Examples of the non-hydrate include alcohol (e.g., methanol, ethanol, n-propanol), dimethylformamide, and the like.

### 4. Myocardium Protective Action

According to the present invention, “ischemia/reperfusion” refers to blood flow that temporarily stops and then restarts. Ischemia/reperfusion is likely to occur when the blood flow is reduced or stopped as occurs in angina or myocardial infarction, when the blood flow is once stopped during surgery and then restarted after the surgery, when the function of heart or liver is lowered, when a side effect of a drug or the symptom of shock by germ infection is induced, or the like.

According to the present invention, “ischemia/reperfusion injury” refers to the injury caused to the blood vessels or tissues by reactive oxygen species which is generated when the blood is reperfused.

According to the present invention, examples of the “reactive oxygen species” include hydroxy radical, superoxide radical, ozone, lipid peroxide, and the like. It is considered that a reactive oxygen species is generated in the body when one suffers from angina or myocardial infarction, when the blood flow is temporarily stopped by surgery or anemia, when one plays sports, when one is exposed to intense ultraviolet or radioactive rays, when one is stressed, when one is infected with germs, or the like.

According to the present invention, “protection against ischemia/reperfusion injury” means protecting cells against an injury caused by ischemia/reperfusion. It is known that hydroxy radicals or superoxide radicals are specifically generated in the heart by ischemia/reperfusion. The serofendic acid according to the present invention is considered to suppress the expression of the hydroxy radical or superoxide radical and thus protect cells against cell disorders caused by the reactive oxygen species induced by ischemia/reperfusion.

According to the present invention, the protective action on cardiac myocytes can be evaluated by morphological observation by various staining techniques or analysis by a cell sorter. The staining technique may be, for example, TUNEL staining, DAPI staining, TMRE fluorescent staining, DCF fluorescent staining, rhod-2 fluorescent staining, or the like (see the examples for the details), but is not limited to these.

The major findings of the present invention are as follows.

(1) In isolated cardiac myocytes, SFA suppressed the cell death induced by H₂O₂ by preserving the ΔΨₘ level in a concentration-dependent manner. The preservation of mitochondrial integrity was most likely achieved by the partial inhibition of [Ca²⁺]ₘ overload and ROS accumulation. An SFA-containing compound represented by the formula (Ia) suppresses ROS like SFA, and therefore may be considered to be capable of protecting cardiac myocytes.

(2) SFA and mitoK_{ATP} opener exhibited comparable protective effects. On the other hand, mitoK_{ATP} channel blocker 5-HD canceled the protective effect of SFA. These observations suggest that SFA acts either directly on the mitoK_{ATP} channel, or upstream of the mitoK_{ATP} channel.

Previous studies in vitro indicated that SFA has neuroprotective effects, as evidenced by the prevention of acute glutamate neurotoxicity in cultured cortical neurons (Taguchi R, et al., Eur J. Pharmacol. 2003; 477:195-203), and the attenuation of ROS-induced oxidative stress in cultured striatal neurons (Osakada F, et al., J Pharmacol Exp Ther. 2004; 311:51-9). Suppression of intracellular ROS generation may constitute an important mechanism of the neuroprotective actions of SFA, since the SFA compound exhibits hydroxyl radical-scavenging activity in electron spin resonance analysis (Kume T, et al., Proc Natl Acad Sci USA. 2002; 99:3288-93).

In recent studies, ROS generation and [Ca²⁺]ₘ overload have been proposed to explain the pathogenesis of ischemia/reperfusion injury of the heart (Weiss J N, et al., Circ Res. 2003; 93:292-301; Griendling K K, Alexander R W. Circulation. 1997; 96:3264-5). ROS and [Ca²⁺]ₘ are the most important inducers of MPTP opening. The present inventors found that SFA suppresses the MPTP opening and partially suppresses the increase of [Ca²⁺]ₘ and ROS.

Recently, the present inventors reported that oxidant stress produces a stereotyped progression of cellular changes in cardiac myocytes (Akao M, et al., Circ Res. 2003; 92:186-94). The first phase we call “priming”: mitochondria undergo [Ca²⁺]ₘ-dependent morphological changes, but ΔΨₘ remains unchanged. Next follows a sudden dissipation of ΔΨₘ mediated by the opening of MPTP (“depolarization” phase); eventually, cells break up into smaller fragments (“fragmentation” phase).

SFA markedly decreased the likelihood that cells would undergo priming: [Ca²⁺]ₘ overload was attenuated and consequently, many mitochondria sufficiently preserved ΔΨₘ. SFA not only decreased the number of cells undergoing ΔΨₘ depolarization, but also delayed the onset of ΔΨₘ loss, to exhibit protective effect on the cardiac myocytes. This mode of action of SFA is equivalent to that of the mitoK_{ATP} channel opener diazoxide (Akao M, et al., Circ Res. 2003; 92:195-202), raising the possibility that the cytoprotective effects of SFA are, directly or indirectly, mediated by the mitoK_{ATP} channel.

5. Cardiac Myocyte Protective Agent and Cardiac Disease Prophylactic and/or Therapeutic Agent

A cardiac myocyte protective agent and a cardiac disease prophylactic and/or therapeutic agent according to the present invention is usable for the purpose of treating cardiac ischemia/reperfusion injury and protecting against myocardial disorder caused by reactive oxygen species. A cardiac myocyte protective agent and a cardiac disease prophylactic and/or therapeutic agent according to the present invention is also applicable to circulatory system diseases such as, for example, cardiomyopathy, heart failure, angina, myocardial infarction, and the like.

“Prophylaxis and/or therapy” generally means obtaining a desired pharmacological effect and/or physiological effect. Such an effect is prophylactic in terms of completely or partially preventing a disease and/or symptom, and is therapeutic in terms of partially or completely curing a disease or an adverse influence caused by the disease. Herein, the term “therapy” means any therapy of a disease of a mammal as a patient, especially, a human, and also encompasses the general meaning of therapy described above. “Therapy” includes, for example, (a) to (c) below.

(a) To prevent a patient, who may have a factor of a disease or symptom but has not been diagnosed as having the factor, from having the disease or symptom;
(b) To block the symptom of a disease, i.e., to inhibit or delay the progress thereof; and
(c) To alleviate the symptom of a disease, i.e., to cause regression or extinction of a disease or symptom or inversion of progress of the symptom.

Examples of cardiomyopathy include dilated cardiomyopathy, hypertrophic occlusive cardiomyopathy, hypertrophic non-occlusive cardiomyopathy, idiopathic cardiomyopathy, restrictive cardiomyopathy, diabetic cardiomyopathy, and the like.

Examples of heart failure include chronic heart failure, chronic congestive heart failure, acute congestive heart failure, acute heart failure, cardiac decompensation, left heart failure, congestive heart failure, acute congestive heart failure, metabolic heart failure, dilated heart failure, high output heart failure, low output heart failure, intractable heart failure, heart failure occurring during recuperation from myocardial infarction, and the like.

The present invention also provides a therapeutic method for cardiac diseases, and a prophylactic and/or therapeutic method for cardiac ischemia/reperfusion injury, each of which comprises administering, to a human, a compound represented by the formula (Ia), a pharmaceutically acceptable salt, or a solvate thereof as a prophylactic and/or therapeutic agent according to the present invention. Where a compound represented by the formula (Ia) (preferably, serofendic acid) is used as a protective agent for cardiac myocyte disorders, the cells are protected against the disorders caused by reactive oxygen species.

A compound represented by the formula (Ia) may form a pharmaceutically acceptable salt or ester. Such a compound is not limited to containing only purified components, but may contain crudely purified components.

A compound represented by the formula (Ia) (e.g., serofendic acid), a pharmaceutically acceptable salt thereof, or a solvate thereof may be administered to a human or a non-human mammal in various forms, either orally or parenterally (e.g., intravenous injection, intramuscular injection, subcutaneous injection, rectal administration, percutaneous administration). A compound represented by the formula (Ia) (e.g., serofendic acid), a pharmaceutically acceptable salt thereof, or a solvate thereof may be used independently, or may be formulated into an appropriate form using a pharmaceutical carrier by a method generally used in accordance with the administration route.

Preferable examples of the form of formulation include oral formulation such as tablet, powdered drug, fine granule, granule, coated tablet, capsule, syrup, troche, or the like; and parental formulation such as inhalant, suppository, formulation for injection (including drip), ointment, eye drop, ophthalmic ointment, nose drop, ear drop, patch, poultice, lotion, liposome, or the like.

Examples of the carrier usable for producing such a formulation include generally used solvent, excipient, coating agent, binder, disintegrator, lubricant, coloring agent, and corrective, and optionally, stabilizer, emulsifier, absorption enhancer, surfactant, pH adjuster, antiseptic, antioxidant, extender, humectant, surface activator, dispersant, buffering agent, preservative, dissolution aid, suspending agent, thickening agent, soothing agent, isotonizing agent, and the like. A formulation can be produced by a usual method by mixing components which are generally used as materials of pharmaceutical formulations. Examples of usable nontoxic components include animal and vegetable oils including soybean oil, beef tallow, synthetic glyceride, and the like; hydrocarbons including fluidic paraffin, squarane, solid paraffin, and the like; ester oils including octyldodecyl myristate, isopropyl myristate, and the like; higher alcohols including cetostearyl alcohol, behenyl alcohol, and the like; silicone resins; silicone oils; surfactants including polyoxyethylene fatty acid ester, sorbitan fatty acid ester, glycerin fatty acid ester, polyoxyethylenesorbitan fatty acid ester, polyoxyethylene cured castor oil, polyoxyethylene-polyoxypropylene block copolymer, and the like; water soluble polymers including hydroxyethylcellulose, polyacrylic acid, carboxyvinyl polymer, polyethyleneglycol, polyvinylpyrrolidone, methylcellulose, and the like; lower alcohols including ethanol, isopropanol and the like; polyhydric alcohols (polyols) including glycerin, propyleneglycol, dipropyleneglycol, sorbitol, polyethyleneglycol, and the like; sugars including glucose, sucrose, and the like; inorganic powders including silicic anhydride, magnesium aluminum silicate, aluminum silicate, and the like; inorganic salts including sodium chloride, sodium phosphate, and the like; purified water; and the like.

Examples of the usable excipient include lactose, fructose, cornstarch, white sugar, dextrose, mannitol, sorbite, crystalline cellulose, silicon dioxide, and the like. Examples of the usable binder include polyvinyl alcohol, polyvinyl ether, methylcellulose, ethylcellulose, gum arabic, tragacanth, gelatin, shellac, hydroxypropylmethylcellulose, hydroxypropylcellulose, polyvinylpyrrolidone, polypropyleneglycol-polyoxyethylene block copolymer, meglumine, and the like. Examples of the usable disintegrator include starch, agar, powdered gelatin, crystalline cellulose, calcium carbonate, sodium hydrogen carbonate, calcium citrate, dextrin, pectin, carboxymethylcellulose-calcium, and the like. Examples of the usable lubricant include magnesium stearate, talc, polyethyleneglycol, silica, cured vegetable oil, and the like. As the coloring agent, any substance which is permitted to be added to pharmaceutical drugs is usable. Examples of the usable corrective include powdered cocoa, menthol, aromatic, mint oil, borneol, powdered cinnamon bark, and the like. Instead of the above-listed components, a salt thereof or a solvate thereof is also usable.

An oral formulation may be produced as follows, for example. To a compound represented by the formula (Ia), a pharmaceutically acceptable salt thereof, or a solvate thereof, an excipient and optionally, a binder, a disintegrator, a lubricant, a coloring agent, a flavoring agent, or the like are added. The resultant substance is produced into powdered drug, fine granule, granule, tablet, coated tablet, capsule, or the like by a usual method.

The tablet or granule may be coated by a well known method with a coating agent such as carnauba wax, hydroxypropylmethylcellulose, macrogol, hydroxypropylmethylphthalate, celluloseacetatephthalate, white sugar, titanium oxide, sorbitan fatty acid ester, calcium phosphate, or the like.

Specific examples of the carrier usable for producing a syrup formulation include sweeteners including white sugar, dextrose, fructose, and the like; suspending agents including gum arabic, tragacanth, carmellose sodium, methylcellulose, sodium alginate, crystalline cellulose, veegum, and the like; and dispersants including sorbitan fatty acid ester, sodium lauryl sulfate, polysolvate 80, and the like. For producing a syrup formulation, any of corrective, aromatic, preservative, dissolution aid, stabilizer, and the like may be optionally added. The syrup formulation may be provided in the form of dry syrup which can be dissolved or suspended before use.

A formulation for injection is usually prepared by, for example, dissolving a pharmaceutically acceptable salt of a compound represented by the formula (Ia) in distilled water for injection. Optionally, any of dissolution aid, buffering agent, pH adjuster, isotonizing agent, soothing agent, preservative, stabilizer, and the like may be added to produce such a formulation by a usual method.

A formulation for injection may be sterilized by filtration using a filter, adding a sterilizer, or the like. A formulation for injection may be produced in a form which can be prepared before use. Namely, a formulation for injection may be produced as a sterile solid composition by lyophilization or the like, which can be dissolved in sterile distilled water for injection or other solvents before use. Examples of the injection method include intravenous drip, intravenous injection, intramuscular injection, intraperitoneal injection, subcutaneous injection, and intradermal injection. The dose for injection depends on the age of the subject of administration, the administration route, or the number of times of administration, and may be varied in a wide range.

An external formulation may be produced by a usual method with no specific limitation. As the base, any of various materials generally used for pharmaceutical drugs, quasi-pharmaceutical drugs, cosmetics, and the like is usable. Examples of such materials include animal and vegetable oils, mineral oils, ester oils, waxes, higher alcohols, fatty acids, silicone oils, surfactants, phospholipids, alcohols, polyhydric alcohols, water soluble polymers, clay minerals, purified water, and the like. Optionally, any of pH adjuster, antioxidant, chelating agent, antiseptic/fungicide, coloring agent, scenting agent, and the like may be added. In the case of an inhalant, which is to be administered by inhalation, a compound represented by the formula (Ia), a pharmaceutically acceptable salt thereof, or a solvate thereof may be delivered from an inhaler, an atomizer or a pressure pack, or in any other convenient manner for delivering aerosol spray. A pressure pack may contain an appropriate atomizing agent. A compound represented by the formula (Ia), a pharmaceutically acceptable salt thereof, or a solvate thereof may be administered in the form of a dry powder composition or a liquid spray so as to be inhaled. For percutaneous absorption from a patch, it is preferable to administer the so-called free form, which does not form a salt. For topical administration to the epidermis, a compound represented by the formula (Ia) may be formulated as ointment, cream or lotion, or as an active component for percutaneous patch. Ointment and cream may be produced by, for example, combining an aqueous or oleaginous base and an appropriate thickening and/or gelating agent. Lotion may be produced using an aqueous or oleaginous base. In general, one or a plurality of types of emulsifiers, stabilizers, dispesants, suspending agents, thickening agents, and/or coloring agents may be incorporated. A compound represented by the formula (Ia) may be also administered by iontophoresis.

Optionally, any of blood flow promoter, disinfectant, anti-inflammatory, cell activator, vitamins, amino acid, moisturizing agent, keratolytic and other components may also be incorporated. The ratio of each active component with respect to the carrier may be varied in the range of 1 to 90% by weight.

A cardiac myocyte protective agent and a cardiac disease therapeutic agent usable for a method according to the present invention may comprise a compound represented by the formula (Ia), a pharmaceutically acceptable salt thereof, or a solvate thereof, usually at a ratio of 0.5% by weight or greater, preferably at a ratio of 10 to 70% by weight.

A compound represented by the formula (Ia), a pharmaceutically acceptable salt thereof, or a solvate thereof, when being used for the above therapy, is purified to at least 90%, preferably 95% or greater, more preferably 98% or greater, and still more preferably 99% or greater.

For oral administration, the dose of a compound represented by the formula (Ia), a pharmaceutically acceptable salt thereof, or a solvate thereof varies because the dose is selected based on various factors including, for example, the administration route; type of disease; degree of symptom; age, gender, or body weight of the patient; type of salt; specific type of disease; pharmacological information on pharmacokinetic and toxicological features and the like; whether a drug delivery system is used or not; and whether the compound or the like is administered as a part of the combination with other drugs or not. Yet, those skilled in the art can set an appropriate dose. A daily dose for an adult (body weight: 60 kg) is about 0.03 to 1000 mg, preferably about 0.1 to 500 mg, and more preferably about 0.1 to 100 mg. Such a daily dose may be administered once or as being divided to several times. The daily dose is not limited to the above range. A dose for a child may be lower than that for an adult.

In the case of parenteral administration through a patch, a daily dose for an adult (body weight: 60 kg) is preferably about 5 to 50 mg, and more preferably about 10 to 20 mg. A formulation for injection may be produced by dissolving or suspending the compound or the like in a pharmacologically acceptable carrier such as physiological saline or commercially available distilled water for injection so as to obtain a concentration of about 0.1 μg/ml of the carrier to about 10 mg/ml of the carrier. A daily dose of the formulation for injection thus produced for an adult patient requiring treatment (body weight: 60 kg) is about 0.06 to 180 mg, and preferably about 0.18 to 60 mg. Such a daily dose may be administered once or as being divided to several times. A dose for a child may be lower than that for an adult.

The administration method actually used may significantly vary, and may depart from the preferable administration methods described in this specification.

A compound represented by the formula (Ia), a pharmaceutically acceptable salt thereof, or a solvate thereof may be used together with a therapeutic agent for another cardiac disease or a pharmaceutical composition for protecting the cells against disorders caused by reactive oxygen species.

Hereinafter, the present invention will be described more specifically by way of examples. It should be noted that the present invention is not limited to the following examples.

### Example 1

### 1. Materials and Method

(1) Primary Culture of Neonatal Rat Cardiac Ventricular Myocytes

Cardiac ventricular myocytes were prepared from 1-2-day-old Wister rats and cultured as previously described (Akao M, et al., Circ Res. 2001; 88:1267-75). In brief, the hearts were removed, and the ventricles were minced into small fragments, which were digested by trypsin dissociation. The dissociated cells were preplated for 1 hour to enrich the culture with myocytes. The non-adherent myocytes were then plated in plating medium consisting of Dulbecco's Modified Eagle Medium (DMEM) (Nacalai tesque; Kyoto, Japan) supplemented with 5% fetal calf serum, penicillin (100 U/ml), streptomycin (100 mg/ml), and 2 μg/ml vitamin B12. The final myocyte cultures contained >90% cardiac myocytes. The cells were maintained at 37° C. in the presence of 5% CO₂ in a humidified incubator. Bromodeoxyuridine (0.1 mM) was incubated in the medium for the first 3 days after plating to inhibit fibroblast growth. Cultures were then placed in serum-free DMEM containing vitamin B12 and transferrin, 24 hours prior to the drug treatment.

(2) Experimental Protocol

Neonatal rat cardiac myocytes in primary culture were randomly assigned to one of three experimental groups:

i) control group;

ii) incubation with 100 μM H₂O₂ for 60 minutes;

iii) pretreated with 100 μM SFA for 30 minutes, followed by 100 μM H₂O₂ for 60 minutes.

At the beginning of the experiment, culture medium for each group was replaced with fresh serum-free DMEM containing the respective drugs, and the cells were exposed to the drugs during the entire experimental period.

SFA was obtained from Eisai Co., Ltd.

(3) MTS Assay

Cell viability was quantified based on metabolic activity using the MTS assay (Promega; Madison, Wis.). MTS tetrazolium compound is a soluble version of 3-(4,5-dimethylthiazol-2-yl)-2,5-diphenyltetrazolium bromide. It is reduced by living cells into a colored formazan product that is soluble in tissue culture medium. This conversion is accomplished by NADPH or NADH in metabolically active cells at 37° C. The quantity of formazan product is directly proportional to the number of living cells in culture (Zhang H M, et al., Circ Res. 2002; 90:1251-8).

The cultures were incubated in serum-free medium containing 20 μl/well of the MTS tetrazolium compound for 3 hours at 37° C. The absorbance of formazan products was photometrically measured at 490 nm with a microplate reader, ARVOsx (PerkinElmer; Shelton, Wash.). Cell viability was expressed as the percentage of the absorbance measured in the control group.

(4) Terminal Deoxynucleotidyl Transferase-Mediated dUTP Nick End-Labeling (TUNEL) Staining

TUNEL staining was performed according to the manufacturer's protocol (Roche; Indianapolis, Ind.). Fluorescein labels incorporated in nucleotide polymers were detected with a fluorescence microscope (Axioskop 2 plus, Zeiss; Thornwood, N.Y.).

(5) 4′,6-diamidino-2-phenylindole (DAPI) Staining

Cells were stained with the DNA binding dye DAPI (5 μM, Molecular Probes; Eugene, Oreg.). The nuclear morphology of cells were visualized and photographed with the fluorescence microscope.

(6) Loading of Cells with Fluorescent Indicator

To monitor ΔΨₘ, cells were loaded with 100 nM tetramethylrhodamine ethyl ester (TMRE) (Molecular Probes) at 37° C. for 20 minutes. To monitor [Ca²⁺]ₘ, the cells were loaded with 2 μM rhod-2 μM (Molecular Probes) at 37° C. for 30 minutes. We assayed intracellular ROS production using chloromethyl-2,7-dichlorodihydrofluorescein diacetate (DCF) (Molecular Probes). Cells were loaded with 4 μM DCF at 37° C. for 30 minutes, and formation of the oxidized derivative was monitored by the increase of green fluorescence.

(7) Fluorescence-Activated Cell Sorter (FACS) Analysis

For FACS analysis of ΔΨₘ, TMRE-loaded cells were harvested by trypsinization at the end of the experimental protocols, and analyzed using FACSAria (BD Biosciences; San Jose, Calif.) (20,000 cells/sample). The fluorescence intensity of TMRE was monitored at 582 nm (FL-2). The FACS data were analyzed using analysis software (WinMDI; http://facs/scripps.edu/software.html).

(8) Confocal Imaging

Cells plated on 35 mm glass-bottom dishes were maintained at 37° C. in the presence of 5% CO₂ using a heater platform installed on a microscope stage, and were placed in serum-free DMEM. After the desired temperature was reached, time-lapse confocal microscopy was started with 2 minute intervals, using a 20× objective lens. Images were taken with laser scanning confocal microscopy (Zeiss, LSM510). TMRE and rhod-2 AM were excited at 543 nm using a helium/neon laser. DCF was excited at 488 nm using an argon laser.

Twenty-five cells were randomly selected in each scan, and the red or green fluorescence intensity was sequentially monitored.

(9) Image Analysis

Quantitative image analysis was performed using image analysis software (ImageJ; http://rsb.info.nih gov/ij/).

(10) Statistical Analysis

Quantitative data in the MTS assay are presented as the mean±SEM. Multiple comparisons among groups were carried out by one-way ANOVA with Fisher's least significant difference as the post-hoc test. A level of p<0.05 was accepted as statistically significant.

### 2. Results

(1) Morphological Observation by TUNEL Staining and DAPI Staining

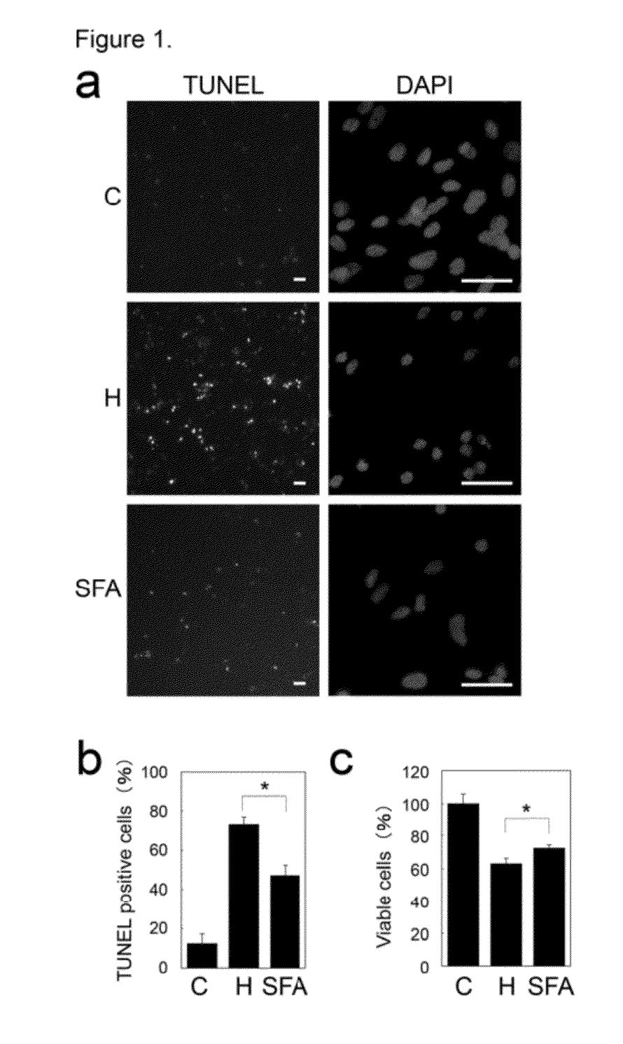

FIG. 1a shows the TUNEL staining and the DAPI staining in each experimental group.

In FIG. 1a, the left panels show TUNEL staining of neonatal rat cardiac myocytes, and the right panels show nuclear counterstaining by DAPI. C=Control cells; H=Cells exposed to 100 μM H₂O₂ for 16 hours; SFA=Cells pretreated with 100 μM SFA followed by 100 μM H₂O₂ for 16 hours. Scale bars: 20 μm.

TUNEL staining detects nuclear DNA strand breaks, which occur during the terminal phase of apoptosis. Control cells exhibited few TUNEL-positive nuclei, but exposure to 100 μM H₂O₂ for 16 hours increased the number of TUNEL-positive nuclei, which can be seen as bright spots, indicating enhanced apoptosis under the treatment with H₂O₂. There were obviously fewer TUNEL-positive nuclei in the SFA-treated group, despite the similar density of cells compared with the H₂O₂ group (FIG. 1a, FIG. 1b). Cells incubated with 100 μM H₂O₂ for 16 hours were also stained with DNA binding dye DAPI. Fragmented or shrunken nuclei were observed in the H₂O₂ group, but SFA displayed a significant protective effect in the preservation of nuclear morphology.

(2) Evaluation of the Cell Survival Ratio

The present inventors examined whether SFA would affect the overall viability of cultured cardiac myocytes exposed to oxidative stress. The MTS assay revealed that 100 μM SFA partly but significantly protected against H₂O₂-induced cytotoxicity (FIG. 1c). FIG. 1c shows cellular viability evaluated by MTS assay. H₂O₂ treatment was 3 hours in this experiment. Data represent mean±SEM (n=13 for each group from two independent cultures). The SFA group significantly protected the cells as compared with the H group (P<0.01).

(3) FACS Analysis by TMRE Fluorescent Staining

The loss of ΔΨₘ is a critical event early in the process of cell death, and has been linked to the opening of MPTP (Weiss J N, et al., Circ Res. 2003; 93:292-301; Crompton M. Biochem J. 1999; 341 (Pt 2): 233-49; Crow M T, et al., Circ Res. 2004; 95:957-70). To examine whether the preservation of ΔΨₘ is associated with the cardioprotective effects of SFA, we assessed the change of TMRE fluorescence by H₂O₂ stimulation in each group using FACS analysis.

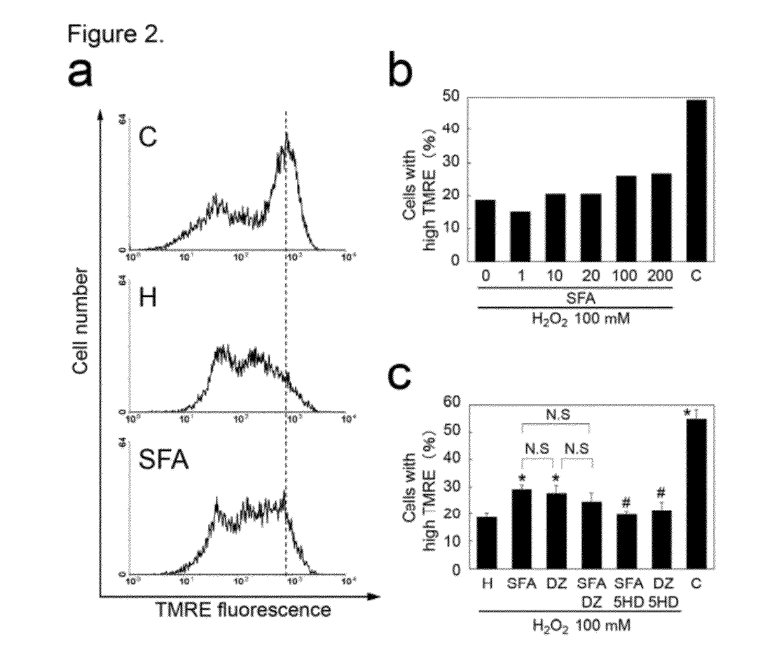

The results are shown in FIG. 2a and FIG. 2b. FIG. 2a shows the FL-2 histograms of FACS data from TMRE-loaded cells. C=Control cells; H=Cells exposed to 100 μM H₂O₂ for 1 hour; SFA=Cells pretreated with 100 μM SFA followed by 100 μM H₂O₂ for 1 hour. In all histograms, the position of the major population of cells in the control group is indicated by a vertical dashed line. These results are representative data from at least 3 independent experiments. FIG. 2b shows the summarized data of the percentage of cells that maintain high (>200) TMRE fluorescence. Cells were pretreated with various concentrations of SFA followed by 100 μM H₂O₂ for 1 hour. C=Control cells. SFA preserved ΔΨₘ in a concentration-dependent manner

A majority of cells in the control group (FIG. 2a, panel C) belonged to a population with a high TMRE fluorescence level (indicated by vertical dashed line). Exposure to H₂O₂ shifted the predominant population to a lower TMRE fluorescence (FIG. 2a, panel H). SFA protected against the H₂O₂-induced loss of ΔΨₘ, preserving a population of cells with a normal ΔΨₘ level (FIG. 2a, panel SFA). These observations were rendered quantitative by plotting the percentage of cells with high TMRE (>200, in this case), as shown in FIG. 2b. Exposure to H₂O₂ for 1 hour resulted in mitochondrial depolarization, whereas SFA prevented the loss of ΔΨₘ in a concentration-dependent manner. The ΔΨₘ-preserving effect of SFA reached its maximum level at 100 μM.

The present inventors further compared the protective effects of SFA with those of diazoxide, a mitoK_{ATP} channel opener. In isolated cardiac myocytes, the present inventors previously reported that diazoxide prevents the loss of ΔΨₘ induced by oxidative stress in a concentration-dependent manner (Akao M, et al., Circ Res. 2001; 88:1267-75).

The results are shown in FIG. 2c and FIG. 2d. FIG. 2c and FIG. 2d show the summarized data of the percentage of cells that maintain high (>200) TMRE fluorescence. The cells were pre-treated with various drugs shown in the figure and then treated with 100 μM H₂O₂ for 1 hour. DZ=100 μM diazoxide; 5HD=500 μM 5-hydroxydecanoate.

As shown in FIG. 2c, the protective effect of 100 μM SFA was comparable to 100 μM diazoxide in preventing the loss of ΔΨₘ induced by 100 μM H₂O₂. The protection afforded by SFA and diazoxide was completely blocked by a mitoK_{ATP} channel blocker, 5-hydroxydecanoate (5HD, 500 μM). 5HD alone did not aggravate the loss of ΔΨₙ, elicited by 100 μM H₂O₂. In another set of experiments, co-application of 100 μM diazoxide and 100 μM SFA did not exhibit an additive effect of SFA alone (FIG. 2d). In addition, SFA alone in the absence of H₂O₂ did not affect the control level of ΔΨₘ(FIG. 2d).

(4) Morphological Observation by TMRE Fluorescent Staining

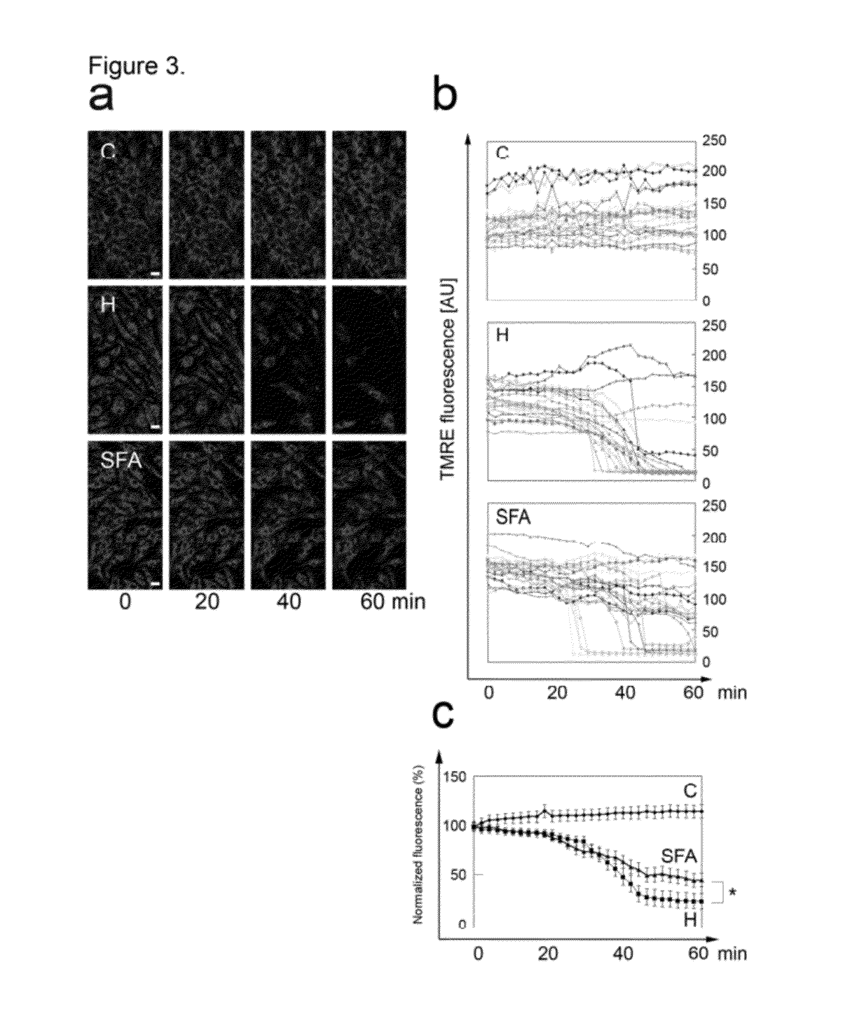

To further confirm the protective effect of SFA in preventing the loss of ΔΨₘ, the present inventors examined the time-dependent changes of ΔΨₘ on a single-cell basis (FIG. 3). Time-lapse confocal analysis of cardiac myocytes loaded with TMRE was performed at 2-minute intervals. Time-lapse scanning began immediately after the application of 50 μM H₂O₂.

The results are shown in FIG. 3. FIG. 3a shows the representative sequential images of TMRE fluorescence in each group. C=Control cells; H=Cells exposed to 100 μM H₂O₂ for 1 hour; SFA=Cells pretreated with 100 μM SFA followed by 100 μM H₂O₂ for 1 hour. Scale bars at 0 min frames: 20 μm.

FIG. 3b shows time course of TMRE fluorescence of 25 cells randomly selected in each group. Similar results were obtained in 3 independent experiments.

FIG. 3c shows mean fluorescence intensity from 25 cells randomly and prospectively selected in each group.

At first, we confirmed that TMRE fluorescence did not change during the 60 minutes of observation in the control group (FIG. 3a, panels C). In contrast, cells treated with H₂O₂ progressively lost their red fluorescence intensity, indicating the irreversible loss of ΔΨₘ (FIG. 3a, panels H). TMRE fluorescence was remarkably preserved in the SFA-treated group (FIG. 3a, panels SFA).

Twenty-five cells were randomly selected in each group, and the TMRE fluorescence intensity from each individual cell was plotted in FIG. 3b.

SFA not only decreased the number of cells undergoing the dissipation of ΔΨₘ, but also delayed the onset of ΔΨₘ loss. FIG. 3c shows the average of TMRE fluorescence intensity from 25 randomly selected cells in each group, indicating the significant protective effects of SFA.

(5) Morphological Observation by DCF Fluorescent Staining

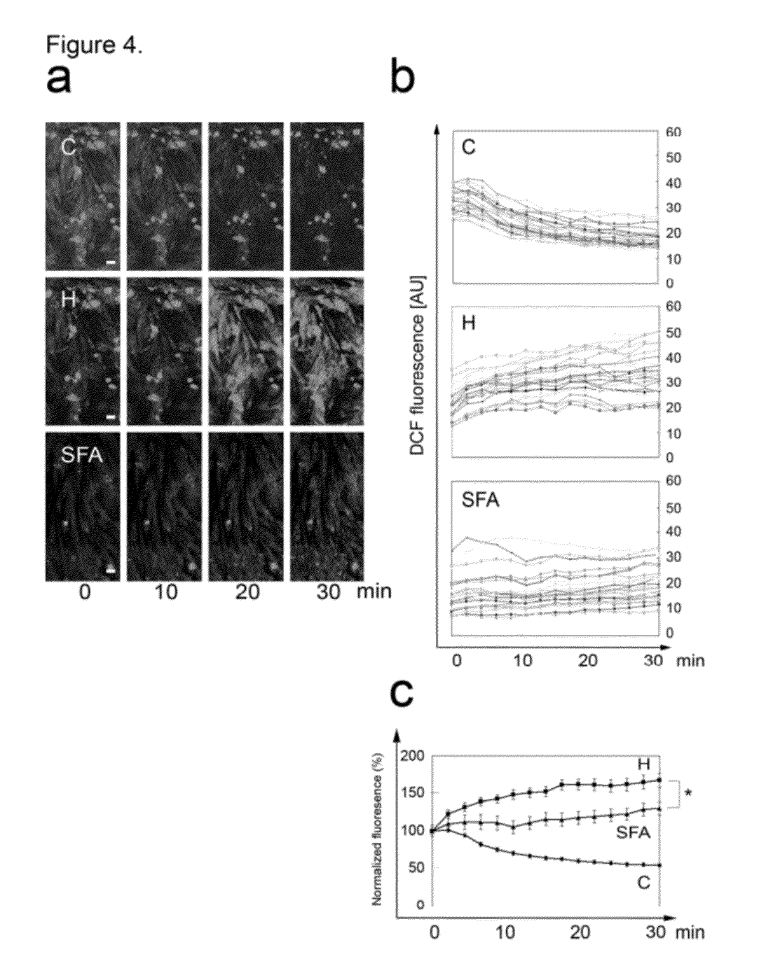

ROS is one of the most important inducers of MPTP opening. To investigate whether the suppression of ROS production is associated with the protective effects of SFA, the present inventors assessed the change of DCF fluorescence by 50 μM H₂O₂ stimulation in each group using time-lapse confocal microscopy (FIG. 4).

The results are shown in FIG. 4a to FIG. 4c. FIG. 4a shows the representative sequential images of DCF fluorescence in each group. C=Control cells; H=Cells exposed to 100 μM H₂O₂ for 30 min; SFA=Cells pretreated with 100 μM SFA followed by 100 μM H₂O₂ for 30 min Scale bars at 0 min frames: 20 μm.

Cells in the control group gradually decreased the DCF fluorescence intensity (FIG. 4a, panels C). In the H₂O₂ group, the DCF fluorescence started to increase progressively upon H₂O₂ application (FIG. 4a, panels H). SFA suppressed the increase in DCF fluorescence (FIG. 4a, panels SFA).

Twenty-five cells were randomly selected from each group, and the DCF fluorescence intensity from each individual cell was plotted (FIG. 4b). The DCF fluorescence intensity of individual cells progressively increased in the H₂O₂ group, but the SFA-treated group blunted the overall increase of DCF fluorescence compared with the H₂O₂ group. Similar results were obtained in 3 independent experiments.

FIG. 4c shows the average of DCF fluorescence intensity from 25 randomly selected cells in each group.

(6) Morphological Observation by rhod-2 Fluorescent Staining

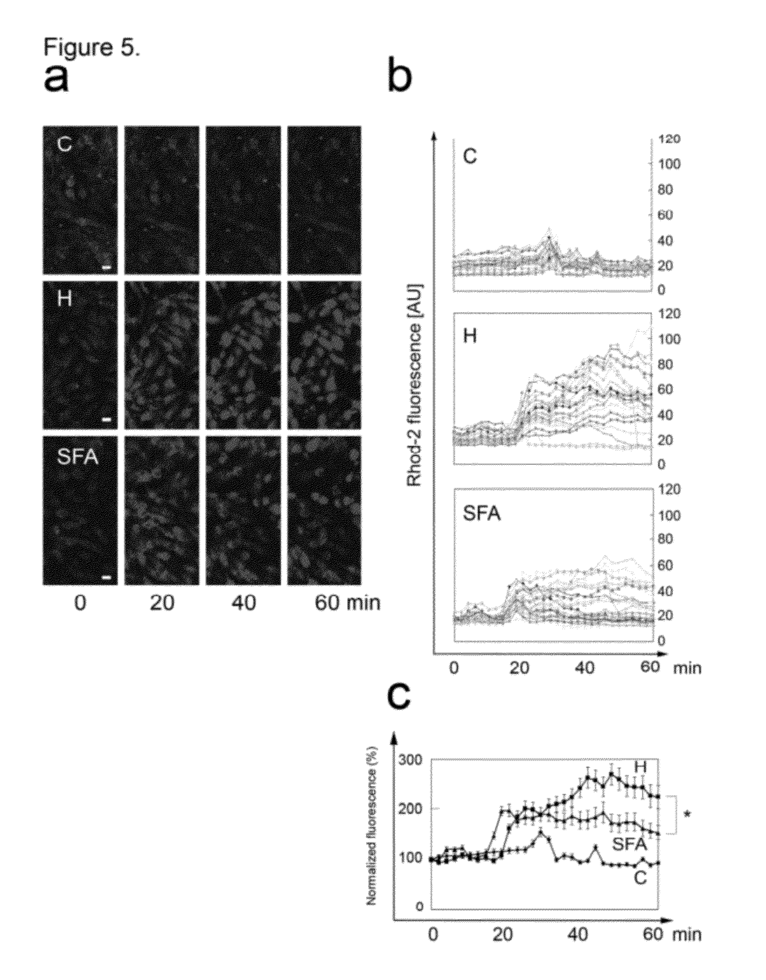

Calcium overload in mitochondrial matrix is one of the critical triggers of cell death and is also an important inducer of MPTP opening. To monitor the [Ca²⁺]ₘ level, the present inventors performed time-lapse confocal microscopy using the [Ca²⁺]ₘ sensitive dye, rhod-2, and observed the change of fluorescence due to 50 μM H₂O₂ stimulation in each group (FIG. 5).

The results are shown in FIG. 5. FIG. 5a shows the representative sequential images of rhod-2 fluorescence in each group. C=Control cells; H=Cells exposed to 100 μM H₂O₂ for 1 hour; SFA=Cells pretreated with 100 μM SFA followed by 100 μM H₂O₂ for 1 hour. Scale bars at 0 min frames: 20 μm.

FIG. 5b shows time course of rhod-2 fluorescence of 25 cells randomly selected in each group. Similar results were obtained in 3 independent experiments.

FIG. 5c shows the mean fluorescence intensity from 25 cells randomly and prospectively selected in each group

We confirmed that the rhod-2 fluorescence did not change during 60 minutes of scanning in the control group (FIG. 5a, panels C). In the H₂O₂ group, rhod-2 fluorescence showed pronounced elevation at approximately 20 minutes after H₂O₂ application, and the elevation persisted thereafter (FIG. 5a, panels H). SFA partly attenuated the [Ca²⁺]ₘ overload observed in the H₂O₂ group (FIG. 5a, panels SFA). FIG. 5b shows the time course of the rhod-2 fluorescence intensity from each individual cell. SFA blunted the overall increase of rhod-2 fluorescence compared with the H₂O₂ group.

### Example 2

### 1. Method

(1) Blood Pressure Measurement

The distal end of the common carotid artery of 8-week-old Sprague-Dawley male rats (about 250 g) was ligated under the effect of pentobarbital anesthetic. Then, to the proximate end thereof, a polyethylene tube filled with heparin-containing saline and connected to a pressure converter was inserted. Changes in blood pressure were recorded by a pen recorder.

(2) Drug Administration to Femoral Vein

A 24-gauge injection needle connected to a syringe via the tube was inserted into the femoral vein, and the liquid drug was injected over 1 to 2 minutes. The blood pressure was monitored from before the liquid drug administration until 10 minutes after the administration.

(3) Drug Administration

Serofendic acid was dissolved in Meylon (7% NaHCO₃) at a concentration of 4 mg/mL. The blood pressure of each individual rat was measured after only the solvent was administered. Then, 10 mg/kg of serofendic acid was administered. The average of the blood pressure for 3 minutes before the administration of each of SFA and Meylon was set as 100%.

### 2. Results

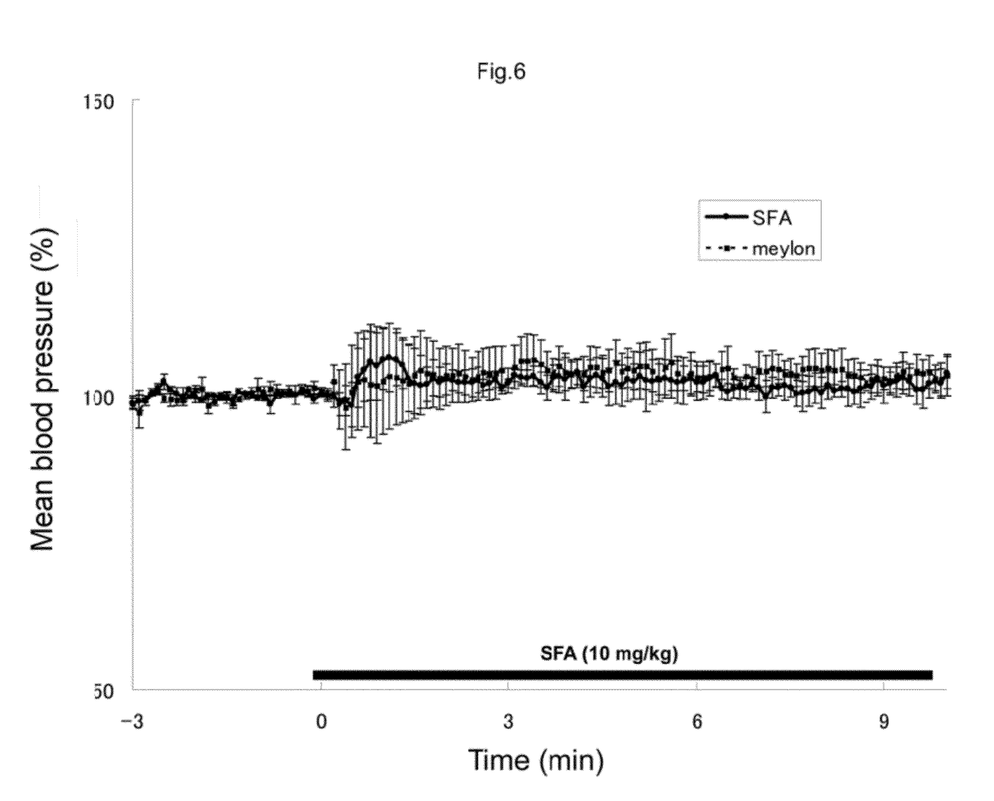

The results are shown in FIG. 6. FIG. 6 shows the action of SFA and Meylon used as a solvent on the average blood pressure of the rats. Even when Meylon as a solvent was administered, no significant change in average blood pressure was observed.

When serofendic acid (10 mg/kg) was also administered, no significant change in average blood pressure was observed, just as in the case of Meylon. This indicates that serofendic acid is a compound which protects cardiac myocytes without the side effect recognized in diazoxide (the action of decreasing blood pressure).

### INDUSTRIAL APPLICABILITY

The present invention provides a cardiac myocyte protective agent, a cardiac disease prophylactic and/or therapeutic agent, and an ischemia/reperfusion injury prophylactic and/or therapeutic agent, each of which comprises a compound represented by the formula (Ia), a pharmaceutically acceptable salt thereof, or a solvate thereof. The present invention also provides a prophylactic and/or therapeutic method for cardiac diseases and cardiac ischemia/reperfusion injury, which uses a compound represented by the formula (Ia), a pharmaceutically acceptable salt thereof, or a solvate thereof. The above compound has a therapeutic effect on heart function disorders and a protective action against ischemia/reperfusion injury, and thus is useful as a cardiac disease therapeutic agent or a protective agent against cardiac ischemia/reperfusion injury.

1. A cardiac myocyte protective agent, comprising a compound represented by the formula (Ia) below, a pharmaceutically acceptable salt thereof, or a solvate thereof: 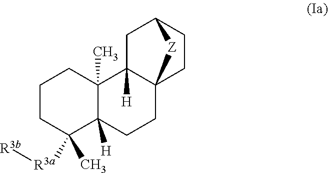
wherein:
Z is a group represented by the following formula: 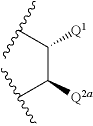
wherein:
Q¹ is represented by the following formula: 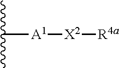
wherein:
A¹ is an optionally substituted C₁₋₆ alkylene group or a single bond;
X² is represented by the formula —S(O)ₘ—, wherein m is an integer of 0, 1, or 2, an oxygen atom, a carbonyl group, or a single bond; and
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
Q^{2a} is a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, or a group represented by the formula —NR^{6a}R^{6b}, wherein R^{6a} and R^{6b} are each independently refer to a hydrogen atom, an optionally substituted C₁₋₆ alkyl group, an optionally substituted C₂₋₆ alkenyl group, an optionally substituted C₂₋₆ alkynyl group, an optionally substituted C₂₋₇ acyl group, or an optionally substituted C₁₋₆ alkylsulfonyl group;
R^{3a} is a carbonyl group, a methylene group, or a single bond; and
R^{3b} is a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, a cyano group, or a group represented by the formula —NR^{5a}R^{5b}, wherein R^{5a} and R^{5b} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group.

2. The protective agent according to claim 1, wherein R^{3a} is a carbonyl group.

3. The protective agent according to claim 1, wherein R^{3b} is a C₁₋₆ alkoxy group or a hydroxyl group.

4. The protective agent according to claim 1, wherein Q^{2a} is a C₁₋₆ alkoxy group or a hydroxyl group.

5. The protective agent according to claim 1, wherein Q¹ is represented by the formula -A¹-S(O)ₘ—R^{4a}, wherein:
A¹ is an optionally substituted C₁₋₆ alkylene group or a single bond;
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
m is an integer of 0, 1, or 2.

6. The protective agent according to claim 1, wherein R^{4a} is an optionally substituted methyl group, an optionally substituted ethyl group, an optionally substituted n-propyl group, or an optionally substituted i-propyl group.

7. The protective agent according to claim 1, wherein m is 1.

8. The protective agent according to claim 1, wherein A¹ is a methylene group.

9. The protective agent according to claim 1, wherein A¹ is a C₁₋₆ alkylene group or a single bond; and R^{4a} is a hydrogen atom, a C₁₋₆ alkyl group, a C₂₋₆ alkenyl group, a C₂₋₆ alkynyl group, a C₆₋₁₄ aromatic hydrocarbon cyclic group, a 5- to 14-membered aromatic heterocyclic group, a C₁₋₆ alkoxy group, or a hydroxyl group.

10. The protective agent according to claim 1, wherein Q^{2a} is a hydrogen atom, a halogen atom, a C₁₋₆ alkyl group, a C₂₋₆ alkenyl group, a C₂₋₆ alkynyl group, a C₁₋₆ alkoxy group, a hydroxyl group, or a group represented by the formula —NR^{6a}R^{6b} wherein R^{6a} and R^{6b} are each independently a hydrogen atom, a C₁₋₆ alkyl group, a C₂₋₆ alkenyl group, a C₂₋₆ alkynyl group, a C₂₋₇ acyl group, or a C₁₋₆ alkylsulfonyl group.

11. The protective agent according to claim 1, wherein the compound represented by the formula (Ia) is serofendic acid.

12. The protective agent according to claim 1, wherein the compound represented by the formula (Ia) is selected from the group consisting of ent-15β-hydroxy-17-methylthio-16α-atisan-19-oic acid, methyl ent-15β-hydroxy-17-methyl sulfinyl-16α-atisan-19-oate, ent-15β-hydroxy-17-methyl sulfonyl-16α-atisan-19-oic acid, ent-15α-hydroxy-17-methylsulfinyl-16β-atisan-19-oic acid, ent-15β,19-dihydroxy-17-methyl sulfinyl-16α-atisane, ent-15β-hydroxy-17-methylsulfinyl-16α-atisane, ent-17-methylsulfinyl-15-oxo-16α-atisan-19-oic acid, ent-15β-hydroxy-17-propyl sulfinyl-16α-atisan-19-oic acid, and ent-17-acetyl-15β-hydroxy-16α-atisan-19-oic acid.

13. A method for the therapeutic treatment and/or prophylaxis of cardiac ischemia/reperfusion injury, comprising administering an effective amount of a protective agent according to claim 1 to a patient in need thereof.

14. A method for the therapeutic treatment and/or prophylaxis of cardiac myocyte disorders caused by reactive oxygen species, comprising administering an effective amount of protective agent according to claim 1 to a patient in need thereof.

15. The method according to claim 14, wherein the reactive oxygen species is a hydroxy radical.

16. A cardiac disease prophylactic and/or therapeutic agent, comprising a compound represented by the formula (Ia) below, a pharmaceutically acceptable salt thereof, or a solvate thereof: 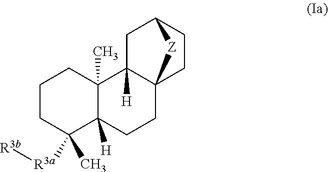
wherein
Z is a group represented by the following formula: 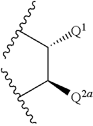
wherein:
Q¹ is represented by the following formula: 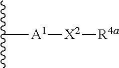
wherein:
A¹ is an optionally substituted C₁₋₆ alkylene group or a single bond;
X² is represented by the formula —S(O)ₘ—, wherein m is an integer of 0, h or 2, an oxygen atom, a carbonyl group or a single bond; and
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group); and
Q^{2a} is a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, or a group represented by the formula —NR^{6a}R^{6b}, wherein R^{6a} and R^{6b} are each independently a hydrogen atom, an optionally substituted C₁₋₆ alkyl group, an optionally substituted C₂₋₆ alkenyl group, an optionally substituted C₂₋₆ alkynyl group, an optionally substituted C₂₋₇ acyl group, or an optionally substituted C₁₋₆ alkylsulfonyl group;
R^{3a} is a carbonyl group, a methylene group or a single bond; and
R^{3b} is a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, a cyano group, or a group represented by the formula —NR^{5a}R^{5b}, wherein R^{5a} and R^{5b} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group; and
a pharmaceutical carrier.

17. The prophylactic and/or therapeutic agent according to claim 16, wherein R^{3a} is a carbonyl group.

18. The prophylactic and/or therapeutic agent according to claim 16, wherein R^{3b} is a hydroxyl group.

19. The prophylactic and/or therapeutic agent according to claim 16, wherein Q^{2a} is a hydroxyl group.

20. The prophylactic and/or therapeutic agent according to claim 16, wherein Q¹ is represented by the formula -A¹-S(O)ₘ—R^{4a}, wherein:
A¹ is an optionally substituted C₁₋₆ alkylene group or a single bond;
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
m is an integer of 0, 1, or 2.

21. The prophylactic and/or therapeutic agent according to claim 16, wherein R^{4a} is an optionally substituted methyl group, an optionally substituted ethyl group, an optionally substituted n-propyl group, or an optionally substituted i-propyl group.

22. The prophylactic and/or therapeutic agent according to claim 16, wherein m is 1.

23. The prophylactic and/or therapeutic agent according to claim 16, wherein A¹ is a methylene group.

24. The prophylactic and/or therapeutic agent according to claim 16, wherein A¹ is a C₁₋₆ alkylene group or a single bond; and R^{4a} is a hydrogen atom, a C₁₋₆ alkyl group, a C₂₋₆ alkenyl group, a C₂₋₆ alkynyl group, a C₆₋₁₄ aromatic hydrocarbon cyclic group, a 5- to 14-membered aromatic heterocyclic group, a C₁₋₆ alkoxy group, or a hydroxyl group.

25. The prophylactic and/or therapeutic agent according to claim 16, wherein Q^{2a} is a hydrogen atom, a halogen atom, a C₁₋₆ alkyl group, a C₂₋₆ alkenyl group, a C₂₋₆ alkynyl group, a C₁₋₆ alkoxy group, a hydroxyl group, or a group represented by the formula —NR^{6a}R^{6b}, wherein R^{6a} and R^{6b} are each independently a hydrogen atom, a C₁₋₆ alkyl group, a C₂₋₆ alkenyl group, a C₂₋₆ alkynyl group, a C₂₋₇ acyl group, or a C₁₋₆ alkylsulfonyl group.

26. The prophylactic and/or therapeutic agent according to claim 16, wherein the compound represented by the formula (Ia) is serofendic acid.

27. The prophylactic and/or therapeutic agent according to claim 16, wherein the compound represented by the formula (Ia) is selected from the group consisting of ent-15β-hydroxy-17-methylthio-16α-atisan-19-oic acid, methyl ent-15β-hydroxy-17-methylsulfinyl-16α-atisan-19-oate, ent-15β-hydroxy-17-methylsulfonyl-16α-atisan-19-oic acid, ent-15α-hydroxy-17-methylsulfinyl-16β-atisan-19-oic acid, ent-15β,19-dihydroxy-17-methylsulfinyl-16α-atisane, ent-15β-hydroxy-17-methyl sulfinyl-16α-atisane, ent-17-methylsulfinyl-15-oxo-16α-atisan-19-oic acid, ent-15β-hydroxy-17-propylsulfinyl-16α-atisan-19-oic acid, and ent-17-acetyl-15β-hydroxy-16α-atisan-19-oic acid.

28. A method for the therapeutic treatment and/or prophylaxis of at least one cardiac disease selected from the group consisting of cardiomyopathy, heart failure, angina, and myocardial infarction, comprising administering an effective amount of a prophylactic and/or therapeutic agent according to claim 16 to a patient in need thereof.

29. A cardiac ischemia/reperfusion injury prophylactic and/or therapeutic agent, comprising a compound represented by the formula (Ia) below, a pharmaceutically acceptable salt thereof, or a solvate thereof: 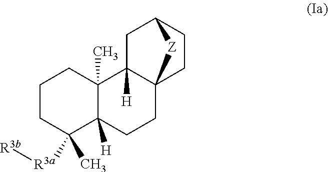
wherein:
Z is a group represented by the following formula: 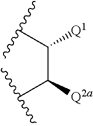
wherein:
Q¹ is represented by the following formula: 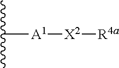
wherein:
A¹ is an optionally substituted C₁₋₆ alkylene group or a single bond;
X¹ is a halogen atom or a cyano group;
X² is represented by the formula S(O)ₘ, wherein m is an integer of 0, 1, or 2, an oxygen atom, a carbonyl group or a single bond;
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group); and
R^{4b} and R^{4c} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group; and
Q^{2a} is a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, or a group represented by the formula —NR^{6a}R^{6b}, wherein R^{6a} and R^{6b} are each independently a hydrogen atom, an optionally substituted C₁₋₆ alkyl group, an optionally substituted C₂₋₆ alkenyl group, an optionally substituted C₂₋₆ alkynyl group, an optionally substituted C₂₋₇ acyl group, or an optionally substituted C₁₋₆ alkylsulfonyl group;
R^{3a} is a carbonyl group, a methylene group or a single bond; and
R^{3b} is a hydrogen atom, a halogen atom, an optionally substituted C₁₋₆ alkyl group, an optionally substituted C₂₋₆ alkenyl group, an optionally substituted C₂₋₆ alkynyl group, an optionally substituted C₁₋₆ alkoxy group, a hydroxyl group, a cyano group, or a group represented by the formula —NR^{5a}R^{5b}, wherein R^{5a} and R^{5b} are each independently a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₂₋₇ acyl group, or an optionally substituted C₁₋₆ alkylsulfonyl group; and
a pharmaceutical carrier.

30. The prophylactic and/or therapeutic agent according to claim 29, wherein R^{3a} is a carbonyl group.

31. The prophylactic and/or therapeutic agent according to claim 29, wherein R^{3b} is a hydroxyl group.

32. The prophylactic and/or therapeutic agent according to claim 29, wherein Q^{2a} is a hydroxyl group.

33. The prophylactic and/or therapeutic agent according to claim 29, wherein Q¹ is a group represented by the formula -A¹-S(O)ₘ—R^{4a}, wherein:
A¹ is an optionally substituted C₁₋₆ alkylene group or a single bond;
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
m is an integer of 0, 1, or 2.

34. The prophylactic and/or therapeutic agent according to claim 29, wherein R^{4a} is an optionally substituted methyl group, an optionally substituted ethyl group, an optionally substituted n-propyl group, or an optionally substituted i-propyl group.

35. The prophylactic and/or therapeutic agent according to claim 29, wherein m is 1.

36. The prophylactic and/or therapeutic agent according to claim 29, wherein A¹ is a methylene group.

37. The prophylactic and/or therapeutic agent according to claim 29, wherein A¹ is a C₁₋₆ alkylene group or a single bond; and R^{4a} is a hydrogen atom, a C₁₋₆ alkyl group, a C₂₋₆ alkenyl group, a C₂₋₆ alkynyl group, a C₆₋₁₄ aromatic hydrocarbon cyclic group, a 5- to 14-membered aromatic heterocyclic group, a C₁₋₆ alkoxy group, or a hydroxyl group.

38. The prophylactic and/or therapeutic agent according to claim 29, wherein Q^{2a} is a hydrogen atom, a halogen atom, a C₁₋₆ alkyl group, a C₂₋₆ alkenyl group, a C₂₋₆ alkynyl group, a C₁₋₆ alkoxy group, a hydroxyl group, or a group represented by the formula —NR^{6a}R^{6b}, wherein R^{6a} and R^{6b} are each independently a hydrogen atom, a C₁₋₆ alkyl group, a C₂₋₆ alkenyl group, a C₂₋₆ alkynyl group, a C₂₋₇ acyl group, or a C₁₋₆ alkylsulfonyl group.

39. The prophylactic and/or therapeutic agent according to claim 29, wherein the compound represented by the formula (Ia) is serofendic acid.

40. The prophylactic and/or therapeutic agent according to claim 29, wherein the compound represented by the formula (Ia) is any one selected from the group consisting of ent-1513-hydroxy-17-methylthio-16α-atisan-19-oic acid, methyl ent-15β-hydroxy-17-methylsulfinyl-16α-atisan-19-oate, ent-15β-hydroxy-17-methylsulfonyl-16α-atisan-19-oic acid, ent-15α-hydroxy-17-methyl sulfinyl-16β-atisan-19-oic acid, ent-15β,19-dihydroxy-17-methylsulfinyl-16α-atisane, ent-15β-hydroxy-17-methylsulfinyl-16α-atisane, ent-17-methyl sulfinyl-15-oxo-16α-atisan-19-oic acid, ent-15β-hydroxy-17-propylsulfinyl-16α-atisan-19-oic acid, and ent-17-acetyl-15β-hydroxy-16α-atisan-19-oic acid.

41. A method of treating a cardiac disorder, comprising administering to a subject in need thereof an effective amount of a compound according to claim 1.

42. The method according to claim 41, wherein R^{3a} is a carbonyl group.

43. The method according to claim 41, wherein R^{3b} is a hydroxyl group.

44. The method according to claim 41, wherein Q^{2a} is a hydroxyl group.

45. The method according to claim 41, wherein Q¹ is represented by the formula -A¹-S(O)ₘ—R^{4a}, wherein:
A¹ is an optionally substituted C₁₋₆ alkylene group or a single bond;
R^{4a} is a hydrogen atom, an optionally substituted C₁₋₆ alkyl group, an optionally substituted C₂₋₆ alkenyl group, an optionally substituted C₂₋₆ alkynyl group, an optionally substituted C₆₋₁₄ aromatic hydrocarbon cyclic group, an optionally substituted 5- to 14-membered aromatic heterocyclic group, an optionally substituted C₁₋₆ alkoxy group, or a hydroxyl group; and
m is an integer of 0, 1, or 2.

46. The method according to claim 41, wherein R^{4a} is an optionally substituted methyl group, an optionally substituted ethyl group, an optionally substituted n-propyl group, or an optionally substituted i-propyl group.

47. The method according to claim 41, wherein m is 1.

48. The method according to claim 41, wherein A¹ is a methylene group.

49. The method according to claim 41, wherein A¹ is a C₁₋₆ alkylene group or a single bond; and R^{4a} is a hydrogen atom, a C₁₋₆ alkyl group, a C₂₋₆ alkenyl group, a C₂₋₆ alkynyl group, a C₆₋₁₄ aromatic hydrocarbon cyclic group, a 5- to 14-membered aromatic heterocyclic group, a C₁₋₆ alkoxy group, or a hydroxyl group.

50. The method according to claim 41, wherein Q^{2a} is a hydrogen atom, a halogen atom, a C₁₋₆ alkyl group, a C₂₋₆ alkenyl group, a C₂₋₆ alkynyl group, a C₁₋₆ alkoxy group, a hydroxyl group, or a group represented by the formula —NR⁶¹), wherein R^{6a} and R^{6b} are each independently a hydrogen atom, a C₁₋₆ alkyl group, a C₂₋₆ alkenyl group, a C₂₋₆ alkynyl group, a C₂₋₇ acyl group, or a C₁₋₆ alkylsulfonyl group.

51. The method according to claim 41, wherein the compound represented by the formula (Ia) is serofendic acid.

52. The method according to claim 41, wherein the compound represented by the formula (Ia) is selected from the group consisting of ent-15β-hydroxy-17-methylthio-16α-atisan-19-oic acid, methyl ent-15β-hydroxy-17-methylsulfinyl-16α-atisan-19-oate, ent-15β-hydroxy-17-methyl sulfonyl-16α-atisan-19-oic acid, ent-15α-hydroxy-17-methylsulfinyl-16β-atisan-19-oic acid, ent-15β,19-dihydroxy-17-methylsulfinyl-16α-atisane, ent-15β-hydroxy-17-methyl sulfinyl-16α-atisane, ent-17-methylsulfinyl-15-oxo-16α-atisan-19-oic acid, ent-15β-hydroxy-17-propylsulfinyl-16α-atisan-19-oic acid, and ent-17-acetyl-15β-hydroxy-16α-atisan-19-oic acid.

53. The method according to claim 41, wherein the cardiac disorder is selected from the group consisting of cardiomyopathy, heart failure, angina, myocardial infarction, and combinations thereof.
